(19) United States
(12) Patent Application Publication
(10) Pub. No.: US 2024/0337666 A1
(43) Pub. Date: Oct. 10, 2024

(54) COMPOSITIONS AND TREATMENTS FOR CORONAVIRUS INFECTIONS

(71) Applicant: Ohio State Innovation Foundation, Columbus, OH (US)

(72) Inventor: Amal AMER, New Albany, OH (US)

(21) Appl. No.: 18/579,084

(22) PCT Filed: Jul. 12, 2022
(86) PCT No.: PCT/US2022/036857

§ 371 Date: Jan. 12, 2024

Related U.S. Application Data
(60) Provisional application No. 63/220,810, filed on Jul. 12, 2021.
(60) Provisional application No. 63/241,467, filed on Sep. 7, 2021.
(60) Provisional application No. 63/248,207, filed on Sep. 24, 2021.

(51) Int. Cl. G01N 33/68; A61K 45/06; A61P 31/14

(52) U.S. Cl. CPC G01N 33/6893; A61K 45/06; A61P 31/14; G01N 33/6884; G01N 2333/165; G01N 2800/12; G01N 2800/26

(57) ABSTRACT

Disclosed are methods for assessing the severity of a coronavirus infection through the measurement of caspase 4 and methods of treating a coronavirus infection comprising administering to an infected subject an inhibitor of CASP4 or its downstream effectors IL-1 B, Cxcl1, or Willebrand factor (vWF). The coronaviral infection may be a SARS-CoV-2 variant.

### CROSS-REFERENCE TO RELATED APPLICATIONS

This application claims benefit of U.S. Provisional Application No. 63/220,810 filed Jul. 12, 2021, U.S. Provisional Application No. 63/241,467 filed Sep. 7, 2021, and U.S. Provisional Application No. 63/248,207, filed Sep. 24, 2021, which are hereby incorporated by reference herein in their entireties.

### STATEMENT OF GOVERNMENT SUPPORT

This invention was made with government support under grant/contract numbers R01 AI124121 and R01 HL127651 awarded by the National Institutes of Health. The government has certain rights in the invention.

### BACKGROUND

Severe acute respiratory syndrome coronavirus 2 (SARS-CoV-2) is the causative infectious agent of the worldwide COVID-19 pandemic. SARS-CoV-2 is a positive sense single-stranded RNA virus that can induce hyper-inflammatory responses, including cytokine storm, in the lungs as well as extra-pulmonary organs in severe cases IL-6, CXCL1, IL-1α, IL-1β and type I interferons, among other cytokines, are thought to contribute to pathological manifestations of the SARS-CoV-2 infection. In addition, formation of thrombi that can cause myocardial infarction, stroke and pulmonary embolism is a hallmark of severe Covid-19. Endothelial and neutrophil dysfunctions during SARS-CoV-2 infection increase the incidence of thromboembolic complications. Thrombus formation is initiated by von Willebrand factor (VWF), a glycoprotein released by damaged endothelial cells and megakaryocytes. VWF also self-associates, forming strings of molecules protruding into the lumen serving as a scaffold for platelet adhesion and aggregation. Cellular sensors of infection, such as Toll-like receptor 2 (TLR2), C-type lectin receptors, and the NLRP3 inflammasome have been implicated in triggering the induction and secretion of cytokines and inflammatory lung damage in SARS-CoV-2 infections. However, the contribution of these pathogen-sensing pathways and other inflammasome components in mediating host defense versus immune-mediated pathology and thrombosis during SARS-CoV-2 infection in vivo remains unclear. While effector molecules downstream of infection-sensing pathways, such as specific inflammatory cytokines, have been targeted in attempts to limit virus-induced tissue damage, most of these strategies failed to exert major benefits in human clinical trials. Therefore, what are needed are new upstream targeting strategies.

### SUMMARY

Disclosed are methods and compositions related to assessing the severity of a coronavirus infection and methods of treating the same.

In one aspect, disclosed herein are methods of treating, inhibiting, reducing, decreasing, and/or ameliorating a coronaviral infection (such as, for example a SARS-CoV-2 (including, but not limited to the SARS-CoV-2 B1.351 variant, SARS-CoV-2B.1.1.7 (alpha), SARS-CoV-2B.1.1.7 variant mutant N501Y (alpha), SARS-CoV-2 delta variant, SARS-CoV-2 P.1 variant, SARS-CoV-2 with T487K, P681R, and L452R mutations in B.1.617.2 (Delta), SARS-CoV-2 with K417N mutation in AY.1/AY.2 (Delta plus), SARS-CoV-2 with D614G, P681H, and D950N mutations in B.1.621 (Mu), SARS-CoV-2 with G75V, T76I, Δ246-252, L452Q, F490S, D614G, and T859N mutations in C.37 (Lambda), SARS-CoV-2 with T478K, Q498R, and H655Y mutations in B.1.1.529 (Omicron)) or the symptoms (thrombosis, pneumonia, inflammation (including, but not limited to inflammatory long damage), and tissue damage) or disease state (such as, for example, COVID-19) thereof comprising administering to a subject with a coronaviral infection an inhibitor of caspase 4 (CASP4)(such as, for example, an anti-CASP4 antibody, small molecule, protein, or, or an antisense oligonucleotide, an siRNA or shRNA that binds to and inhibits Casp4.

Also disclosed herein are methods of assessing the severity of a coronaviral infection (such as, for example, a SARS-CoV-2 infection including, but not limited to the SARS-CoV-2 B1.351 variant, SARS-CoV-2B.1.1.7 (alpha), SARS-CoV-2B.1.1.7 variant mutant N501Y (alpha), SARS-CoV-2 delta variant, SARS-CoV-2 P.1 variant, SARS-CoV-2 with T487K, P681R, and L452R mutations in B.1.617.2 (Delta), SARS-CoV-2 with K417N mutation in AY.1/AY.2 (Delta plus), SARS-CoV-2 with D614G, P681H, and D950N mutations in B.1.621 (Mu), SARS-CoV-2 with G75V, T76I, Δ246-252, L452Q, F490S, D614G, and T859N mutations in C.37 (Lambda), SARS-CoV-2 with T478K, Q498R, and H655Y mutations in B.1.1.529 (Omicron)) comprising: a) obtaining a pulmonary tissue sample from the subject; and b) measuring the level of CASP4 expression (including, but not limited to performing western blot, quantitative western blot, RNA sequencing (RNAseq), Single Cell RNA Sequencing, (scRNAseq), high-performance liquid chromatography (HPLC), liquid chromatography-mass spectrometry (LC/MS), enzyme linked immunosorbent assay (ELISA), immunoelectrophoresis, and/or protein array) in the sample; wherein the level of CASP4 directly correlates with severity of infection.

In one aspect, disclosed herein are methods of assessing the severity of a coronaviral infection of any preceding aspect, further comprising administering to the subject a neutrophil inhibitor (such as for example serpin B), an inhibitor of CASP4, von Willebrand factor (vWF), IL-1β, or Cxcl1 (such as, for example, an antibody (such as, for example anti-CASP4 antibody (such as, for example, 4B9 and/or MA5-26748), an anti-vWF antibody (including, but not limited to AJW200, ARC1779, Caplacizumab (ALX0081), 82D6A3, h6B4-Fab, GPG-290), an anti-IL1β antibody (canakinumab (ILARIS®), rilonacept (ARCALYST®), anakinra (KINERET), gevokizumab, and/or ly2189102), and/or an anti-Cxcl1 antibody (such as, for example, MA5-23745, MAB453-100, MM0208-9A18, and/or HL2401)), small molecule, protein, or an antisense oligonucleotide, an siRNA or shRNA that binds to and inhibits Casp4, I11b, vWF, or Cxcl1 (including, but not limited to siRNAs and shRNAs available for purchase from origene, cellomicstech, Hindawi, thermofisher, vector biolabs, applied biological materials, and ABBEXA®).

### BRIEF DESCRIPTION OF THE DRAWINGS

The accompanying drawings, which are incorporated in and constitute a part of this specification, illustrate several embodiments and together with the description illustrate the disclosed compositions and methods.

FIGS. 1A, 1B, 1C, 1D, 1E, 1F, and IG show that CASP4 is upregulated in humans and mice infected with SARS-CoV-2. FIG. 1A shows CASP4 expression levels from RNA sequencing of nasopharyngeal swab samples from patients with no disease, mild SARS-CoV-2, or severe SARS-CoV-2 [GSE145926]. One way ANOVA with Tukey's multiple comparisons test. FIG. 1B shows human lung samples from 3 donors with healthy lungs or from 3 donors who died of SARS-CoV-2 were stained for CASP4 (brown). Black boxes (i, ii) outline zoomed regions. FIG. 1C shows quantification of CASP4 positive cells from lungs in 1B. unpaired t test. d-f, Mice were infected for 4 days with mouse adapted SARS-CoV-2 (MA10, 10⁵ pfu). FIG. 1D shows Casp11 RNA (green, RNAscope in situ hybridization) and DAPI (blue) were visualized (3D Intensity projection image) in lung sections using 20× objective. FIG. 1E shows Casp11 RNA levels were quantitated in lung samples (N=3) by qRT-PCR, unpaired t test. FIG. 1F shows CASP11 protein levels in lungs (N=3) were examined by Western blot. FIG. 1G shows K18-hACE2 mice were infected for 4 days with human SARS-CoV-2 (WA1, 10⁵ pfu) and Casp11 RNA levels were quantitated in lung samples (N=4) by qRT-PCR, unpaired t test. *p<0.05, **p<0.005, ****p<0.0001.

FIGS. 2A, 2B, 2C, 2D, 2E, and 2F show that Casp11^{-/-} mice show decreased SARS-CoV-2 infection severity without affecting viral titers but by modulating specific inflammatory programs. FIGS. 2A, 2B, and 2C show that WT, Casp11^{-/-}, and Gsdmd^{-/-} mice were infected with SARS-CoV-2 (MA10, 105 pfu). FIG. 2A shows weight loss was tracked for 7 d. *P<0.05, **P<0.005, ****P<0.0001; ANOVA with Bonferroni's multiple comparisons test, day 0 to 4 WT (n=7), Casp11^{-/-} (n=10), Gsdmd^{-/-} (n=9); day 5 to 7 WT (n=4), Casp11^{-/-} (n=7), Gsdmd^{-/-} (n=6). Error bars in represent SEM. FIG. 2B shows TCID50 viral titers were quantified in lung tissue homogenates. Error bars represent SD of the mean. FIG. 2C shows sections from noninfected control lungs or lungs collected at 4 d after infection were stained for SARS-CoV-2 nucleocapsid protein (brown staining, images representative of at least three mice per group). Insets outline zoomed regions. FIGS. 2D, 2E, and 2F show WT, Casp11^{-/-}, and Gsdmd^{-/-} mice (n=3) were infected with SARS-CoV-2 (MA10, 105 pfu) for 2 d. RNA was extracted from lungs and subjected to RNA sequencing. FIG. 2D shows PCA of SARS-CoV-2-infected lung gene expression with points representing individual WT (gray), Casp 11^{-/-} (blue), and Gsdmd^{-/-} (green) mice. PC1 and PC2 represent principal component1 and 2, respectively. FIG. 2E shows Top 30 significant Gene Ontology Biological Pathways are depicted. Node size indicates the number of transcripts within each functional category. Edges connect overlapping gene sets. Numbers represent individual replicates, and color indicates relative up-regulation (red) or down-regulation (blue) in gene expression. TNF: tumor necrosis factor alpha, ROS: reactive oxygen species. FIG. 2F shows a heatmap of significantly changed cytokine and chemokine genes when comparing Casp11^{-/-} infected lungs versus WT. Expression scaling is relative to WT and Gsdmd⁴ mice for comparisons (n=3) (P<0.05).

FIGS. 3A, 3B, 3C, 3D, 3E, 3F, 3G, 3H, 3I, 3J, and 3K show that Casp11^{-/-} mice show decreased lung inflammation, less neutrophil recruitment, and altered neutrophil function in response to SARS-CoV-2 infection. FIGS. 3A and 3B show that WT and Casp11^{-/-} mice were infected with SARS-CoV-2 (MA10, 105 pfu). FIG. 3A shows lung sections from day 4 after infection were stained with H&E to visualize lung damage and airway consolidation. FIG. 3B shows lung sections as in A were analyzed by the color deconvolution method to quantify cellularity as an indicator of cellular infiltration and alveolar wall thickening; ANOVA with Tukey's multiple comparisons test. (FIGS. 3C and 3D show that lung homogenates from 2 or 4 d after infection were analyzed by ELISA for detection of CXCL1, IL-1β, or IL-6; ANOVA with Tukey's multiple comparisons test. FIGS. 3E and 3F show that macrophages were purified from lungs of mice of the indicated genotype. The cells were infected with mouse-adapted SARS-CoV-2 (multiplicity of infection [MOI] 1 for 24 h). Cell supernatants were analyzed by ELISA, or cellular RNA was analyzed by qRT-PCR for the indicated chemokine/cytokines; ANOVA with Tukey's multiple comparisons test. FIG. 3G shows a heatmap of significantly changed neutrophil-related genes comparing Cas11^{-/-} infected lungs versus WT (P<0.05). Expression scaling is relative to WT and Gsdmd^{-/-} mice for comparisons. Numbers represent individual replicates, and color indicates relative up-regulation (red) or down-regulation (blue) in gene expression. FIGS. 3H and 3I show lung sections of day 2 SARS-CoV-2-infected WT, Casp11^{-/-}, and Gsdmd^{-/-}-mice (n=5) stained with neutrophil marker Ly6G (3H) and quantified in 3I. FIGS. 3J and 3K show that flow cytometry of lung single-cell suspensions previously gated on CD45+ cells from WT (n=4), Casp11^{-/-} (n=6), and Gsdmd^{-/-} (n=4) mice (3J) as in H and quantified in 3K. All error bars represent SEM. *P<0.05, **P<0.005.

FIGS. 4A, 4B, 4C, 4D, 4E, 4F, 4G, and 4H show that Casp11^{-/-} neutrophils undergo less NETosis, and Casp11^{-/-} mice show decreased indicators of coagulopathy in lungs after SARS-CoV-2 infection. FIG. 4A shows neutrophils from WT, Casp11^{-/-}, and Gsdmd^{-/-} mice were treated with PMA, and NET formation was visualized by staining with anti-mouse Histone 2b (H2B) (red) and anti-dsDNA (green). Images were captured at 60× magnification. FIG. 4B shows the percentage of cells undergoing NETosis as averaged from 10 fields of view (FOVs) for each experimental replicate. Error bars represent SEM. FIGS. 4C, 4D, 4E, 4F, and 4G show WT, Gsdmd^{-/-}, and Casp11^{-/-} mice were infected with SARS-CoV-2 (MA10, 105 pfu). Lungs were collected at day 4 after infection. FIG. 4C shows RNA for VWF (green) was stained by RNAscope ISH, and nuclei were stained with DAPI (blue). Images were captured by a 20× objective in a 3D stitched panoramic view. Intensity projection images were created using IMARIS software. Scale bars represent 500 μm. FIGS. 4D and 4E show western blotting of lung homogenates from noninfected WT and SARS-CoV-2-infected WT and Casp11 mice (4D) as described in C were quantified in 4E; unpaired t test. Error bars represent SEM. FIG. 4F shows qRT-PCR quantification of KLF2 in the lungs of mice as described in 4C; unpaired t test. Error bars represent SEM. FIG. 4G shows confocal microscopy for the colocalization of VWF RNA (green) with endothelial VEGF receptor subtype 1 (FLT1, red) in the lungs of mice as described in C. Nuclei were stained with DAPI (blue). Images were captured with a 20× objective in a z-stack 3D view and visualized using intensity projection function of IMARIS software. FIG. 4H shows vasculature imaging of intact lungs 4 d after infection. Below: Higher-magnification view of the regions in yellow boxes. (Scale bar, 200 μm.) *P<0.05, **P<0.005.

FIGS. 5A and 5B show cell type-specific expression profiles of CASP4 and CASP5 genes in the human lung. Single cell expression data contained in the Human Protein Atlas version 21.0 was mined to identify cell types in the human lung that express 5A, CASP4 and 5B, CASP5.

FIG. 6 shows human caspase expression levels in nasopharyngeal swab samples. CASP5, CASP7, and CASP10 expression levels from RNA sequencing of nasopharyngeal swab samples from patients with no disease, mild SARS-CoV-2, or severe SARS-CoV-2 [GSE145926], one way ANOVA with Tukey's multiple comparisons test, *P<0.05.

FIGS. 7A, 7B, and 7C show expression profiles of Casp11 in murine lung immune cells. Analysis of single cell gene expression from mock and SARS-CoV-2 infected lungs.

FIG. 7A shows UMAP analysis of cell subsets in mock lungs (left) and lungs infected with SARS-CoV2 (right). FIG. 7B shows log normalized read counts (NRC) of Casp11 in single cells derived from lungs of mock (left) and SARS-CoV-2 infected (right) mice. FIG. 7C shows violin plots of the relative expression of Casp11 across cell types identified in mock (left) and SARS-CoV-2 infected lungs (right).

FIGS. 8A, 8B, 8C, 8D, 8E, and 8F show changes in inflammatory responses in Casp11^{-/-} and Gsdmd^{-/-} SARS-CoV-2 infected lungs. FIG. 8A shows a heat map of significant gene expression changes (p-value<0.05) in infected Casp11^{-/-} and Gsdmd^{-/-} lungs. FIGS. 8B and 8C show statistical analysis of ISG expression in Casp11^{-/-} and Gsdmd^{-/-} infected lungs relative to WT. Each point represents transcripts within the dataset. The top 300 IFNb-responsive ISGs are highlighted in black. Dashed lines represent LFC and p-value cutoffs (LFC 10.581 and p-value 0.05). FIG. 8D shows functional enrichment analysis of 236 downregulated genes in Casp11^{-/-} SARS-CoV-2-infected lungs relative to infected WT. Red horizontal line represents threshold of significance p-value 0.05. FIG. 8E shows functional enrichment analysis of 224 downregulated genes in Gsdmd infected lungs relative to WT infection. Red horizontal line represents threshold of significance p-value 0.05. FIG. 8F shows functional enrichment analysis of 328 upregulated genes in Casp11^{-/-} infected lungs relative to WT infection. Bar graphs represent the top 10 significantly enriched Gene Ontology Biological Processes. Red horizontal line represents threshold of significance p-value 0.05.

FIG. 9 shows that WT and Casp11^{-/-} mice were infected with mouse adapted SARS-CoV-2 (10⁵ pfu). Lungs were collected at day 4 post infection. Lung tissue was sectioned and stained for IL-1β (green), and DAPI (blue).

FIGS. 10A and 10B show Casp11−/− neutrophils are impaired in NET formation. FIG. 10A shows neutrophils from WT, Casp11^{-/-} and Gsdmd^{-/-} mice were treated with supernatants of SARS-CoV-2-infected epithelial cells from WT, Casp11^{-/-} and Gsdmd^{-/-} mice and NET formation was stained with anti-mouse Histone 2b (red), anti-dsDNA (green). Images were captured at 60× magnification. FIG. 10B shows the percentage of cells undergoing NETosis from images as in 10A averaged from 10 fields of view (FOV) for each experimental replicate, one way ANOVA with Tukey's multiple comparisons test, *P<0.05, **P<0.005.

FIG. 11 shows that RNA of VWF (green) was stained by RNAscope in situ hybridization (ISH), and nuclei are showed in DAPI (blue). Images were captured by a 20× objective.

FIG. 12 shows quantification of ISH RNAscope staining of endothelial VEGF receptor subtype 1(FLT1) in lung sections. Mice were infected with mouse adapted SARS-CoV-2 (10⁵ pfu). Lungs were collected at day 4 post infection. Original Images were captured by a 20× objective in a 3D stitched panoramic view showing the whole lung section in x, y and Z. DAPI and FLT1 mRNA spots and were quantified by using the spot function in IMARIS software. Unpaired t test.

FIG. 13 shows a representative photograph of lungs with and without tissue clearing.

FIG. 14 shows a cartoon showing that Casp11-mediates hyperinflammation, neutrophil infiltration, NETosis, thrombus formation and vascular damage during SARS-CoV-2 infection.

### DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. METHODS OF TREATING CORONAVIRAL INFECTIONS

SARS-CoV-2 is a positive sense single-stranded RNA virus that can induce hyper-inflammatory responses, including cytokine storm manifested by high production of IL-1p, IL-6, IL-8 and other cytokines and chemokines. Critically ill SARS-CoV2-infected patients suffer from acute respiratory failure, circulatory shock, and thrombotic complications, including cerebrovascular accident, and myocardial infarction, as well as a hyperinflammatory state that are all associated with higher incidence of long-Covid. The deterioration of lung function has been attributed to a maladaptive immune response rather than increased viral loads.

SARS-CoV-2 enters the airway epithelial cells after binding to angiotensin-converting enzyme 2 (ACE2) with its spike (S) protein. Endothelial cells also express high levels of ACE2 and viral infection can disrupt endothelial cell function directly or by evoking an inflammatory response resulting in the release of plasminogen activator inhibitor-1 (PAI-1) which, along with complement C5a-induced release of tissue factor (TF) which exacerbates thrombosis. These events augment complement-coagulation pathway interaction causing endothelial injury, hypercoagulability, stroke, and thrombotic microangiopathies.

Emerging data in clinical studies indicate that complement activation plays a critical role in pathogenesis and disease severity of SARS-CoV-2. The contribution of complement in SARS-CoV-2-assocated pathology was also demonstrated in C3-deficient mice which experienced reduced respiratory dysfunction, limited cellular infiltration, and lower levels of cytokines in the lungs and the blood. Members of the complement system circulate in plasma and are expressed on cellular surfaces. They are activated through three pathways: classical, lectin and alternative. Activated complement promotes inflammation with C3a and C5a, and direct cell lysis with the assembly of the membrane attack complex (MAC) C5b-9. Whereas C3a stimulates platelet activation and aggregation, the activation of C5 (C5a) is responsible for the expression of tissue factor (TF) on activated leukocytes and endothelial cells. Endothelial injury and activated platelets generate von Willebrand Factor (vWF) that self-associates, forming strings serving as a scaffold for platelet adhesion and aggregation. With the help of neutrophil extracellular traps (NETs) formed through NETosis, thrombosis is further promoted. Neutrophils and monocytes prominently express the C5aR on their cell surface, and contribute to cytokine production including IL-1β, IL-6 and IL-8. Then, the process of thrombosis is attacked by active plasmin which cleaves fibrin to D-dimers. Notably, severe COVID-19 has been characterized with increased D-dimer levels in the blood and has been adopted as a biomarker for coagulopathy. In most pathophysiological situations including SARS-CoV2 infection, the activation of both the complement and coagulation cascades occurs simultaneously. Lung biopsy samples from patients with severe COVID-19 showed widespread C3a generation and C3-fragment deposition, NET complexes and prominent increase of serum C5a. This pathophysiological course of events is consistent with the finding of microcirculatory clot formation in the postmortem sections of patients with COVID-19 and is often associated with cardio- and cerebro-vascular complications, myocardial ischemia, and micro- and macro-circulatory thromboembolic complications.

Few emerging studies reported the subtle role of the canonical inflammasome in COVID-19, yet no studies examined the contribution of the non-canonical inflammasome. Caspase-11 (CASP11) (mouse homolog of human CASP4) is an enzyme critical for defense against bacterial pathogens and is a component of the non-canonical inflammasome. (both human CASP4 and mouse CASP11 will be referred to as CASP4/11 to avoid confusion). CASP4/11 is weakly expressed in resting cells and is induced in response to several signals. Importantly, Casp4/11) is highly expressed in the airway of SARS-CoV-2-infected patients, and its expression levels increase with disease severity. Here, we investigate the role of a major member of the non-canonical inflammasome CASP4/11, and its downstream effector Gasdermin D (GSDMD) in SARS-CoV-2 infection and disease severity using knockout mouse models and mouse-adapted SARS-CoV-2. Additionally, we found that human lung sections from COVID-19 patients show higher levels of CASP4 staining compared with healthy lung controls. Thus, in one aspect, disclosed herein are methods of assessing the severity of a coronaviral infection (such as, for example, a SARS-CoV-2 infection including, but not limited to the SARS-CoV-2 B1.351 variant, SARS-CoV-2B1.1.1.7 (alpha), SARS-CoV-2B.1.1.7 variant mutant N501Y (alpha), SARS-CoV-2 delta variant, SARS-CoV-2 P.1 variant, SARS-CoV-2 with T487K, P681R, and L452R mutations in B.1.617.2 (Delta), SARS-CoV-2 with K417N mutation in AY.1/AY.2 (Delta plus), SARS-CoV-2 with D614G, P681H, and D950N mutations in B.1.621 (Mu), SARS-CoV-2 with G75V, T76I, Δ246-252, L452Q, F490S, D614G, and T859N mutations in C.37 (Lambda), SARS-CoV-2 with T478K, Q498R, and H655Y mutations in B.1.1.529 (Omicron)) comprising: a) obtaining a pulmonary tissue sample from the subject; and b) measuring the level of CASP4 expression (including, but not limited to performing western blot, quantitative western blot, RNA sequencing (RNAseq), Single Cell RNA Sequencing, (scRNAseq), high-performance liquid chromatography (HPLC), liquid chromatography-mass spectrometry (LC/MS), enzyme linked immunosorbent assay (ELISA), immunoelectrophoresis, and/or protein array) in the sample; wherein the level of CASP4 directly correlates with severity of infection.

It is understood and herein contemplated that the disclosed methods of assessing the severity of a coronaviral infection are not limited to a particular coronaviral infection (such as, SARS-Cov-2), but can be performed on infected tissue samples from a subject infected with any coronavirus including but not limited to avian coronavirus (IBV), porcine coronavirus HKU15 (PorCoV HKU15), Porcine epidemic diarrhea virus (PEDV), HCoV-229E, HCoV-OC43, HCoV-HKU1, HCoV-NL63, SARS-CoV, SARS-CoV-2 (including, but not limited to the SARS-CoV-2 B1.351 variant, SARS-CoV-2B.1.1.7 (alpha), SARS-CoV-2B.1.1.7 variant mutant N501Y (alpha), SARS-CoV-2 delta variant, SARS-CoV-2 P.1 variant, SARS-CoV-2 with T487K, P681R, and L452R mutations in B.1.617.2 (Delta), SARS-CoV-2 with K417N mutation in AY.1/AY.2 (Delta plus), SARS-CoV-2 with D614G, P681H, and D950N mutations in B.1.621 (Mu), SARS-CoV-2 with G75V, T76I, Δ246-252, L452Q, F490S, D614G, and T859N mutations in C.37 (Lambda), SARS-CoV-2 with T478K, Q498R, and H655Y mutations in B.1.1.529 (Omicron)), and MERS-CoV.

Detection of a coronaviral infection and/or assessment of severity can lead a practicing physician to treat the subject source of the biological sample tested. Accordingly, in one aspect, disclosed herein are methods of assessing the severity of a coronaviral infection, further comprising administering to the subject a neutrophil inhibitor (such as for example serpin B) or an inhibitor of CASP4, von Willebrand factor (vWF), IL-1β, or Cxcl1. Inhibitors of CASP4, vWF, IL-1b, and Cxcl1 can include antibodies, antisense oligonucleotides, small interfering RNA (siRNA), short hairpin RNA (shRNA), proteins, and small molecules. For example, the antibody can be an anti-CASP4 antibody (such as, for example, 4B9 and/or MA5-26748), an anti-vWF antibody (including, but not limited to AJW200, ARC1779, Caplacizumab (ALX0081), 82D6A3, h6B4-Fab, GPG-290), an anti-IL1β antibody (canakinumab (ILARIS®), rilonacept (ARCALYST®), anakinra (KINERET), gevokizumab, and/or ly2189102), and/or an anti-Cxcl1 antibody (such as, for example, MA5-23745, MAB453-100, MM0208-9A18, and/or HL2401. Where antisense oligonucleotides, siRNAs, or shRNAs are used, the siRNA and/or shRNA can be obtained from commercially available sources such as origene, cellomicstech, Hindawi, thermofisher, vector biolabs, applied biological materials, and ABBEXA®. For example, an anti-Casp4 siRNA can be obtained from thermofisher, applied biological materials, origene; an anti-Casp4 shRNA can be obtained from vector biolabs, origene, ABBEXA®; an anti-Cxcl1 siRNA can be obtained from thermofisher or applied biological materials; an anti-Cxcl1 shRNA can be obtained from vector biolabs, origene, cellomicstech, ABBEXA®; an anti-i11b siRNA can be obtained from ABBEXA® or Hindawi; and an anti-i11b shRNA can be obtained from vector biolabs, origene, ABBEXA®.

In one aspect, the subject is not limited to a human subject, but can be a non-human primate, mouse, rat, pig, goat, cow, dog, cat, horse, sheep, or bird (including, but not limited to chicken, turkey, duck, quail, pigeon, or pheasant).

Detection of protein or RNA expression can be achieved by any means known in the art for determining protein or RNA expression levels including, but not limited to western blot, quantitative western blot, RNA sequencing (RNAseq), Single Cell RNA Sequencing, (scRNAseq), high-performance liquid chromatography (HPLC), liquid chromatography-mass spectrometry (LC/MS), enzyme linked immunosorbent assay (ELISA), immunoelectrophoresis, and/or protein array.

One major role for CASP4/11 is the cleavage of GSDMD. Once cleaved, the GSDMD N-terminal fragment inserts into the plasma membrane of eukaryotic cells to form pores that allow the release of IL-1β and other molecules, sometimes leading to cell lysis and death known as pyroptosis. As shown herein, CASP4/11-mediated functions are not executed by GSDMD which is a major effector of CASP4/11. Instead, the expression of other effectors related to inflammation, complement and thrombosis is dysregulated in a CASP4/11-dependent manner upon SARS-CoV-2 infection. which was revealed through our global transcriptomic analysis of lungs from WT versus Casp4/11^{-/-} mice infected with SARS-CoV-2. Hence, CASP4/11 controls the expression of specific cytokines, inflammatory receptors, cell migration molecules, pro-thrombotic factors, and members of the complement cascade during SARS-CoV-2 infection. Notably, the expression of IFN-stimulated genes (ISGs), which are abundantly upregulated by type I IFN stimulation in murine airway epithelial cells during SARS-CoV-2 infection were not affected by the absence of CASP11/4.

Importantly, we found that CASP4/11 is required for the accumulation of VWF in the lungs during SARS-CoV-2 infection which is a sign of endothelial injury. Vascular tracing revealed distinctive vascular features in WT SARS-CoV-2-infected lungs with pronounced vascular thickening and obliteration. In stark contrast, Casp4/11^{-/-} infected lung vasculature did not show these abnormalities, confirming less endothelial damage/dysfunction. On the other hand, the expression of Kruppel-Like Factor 2 (KLF2) which is an endothelial protective transcription factor is significantly reduced after SARS-CoV-2-infection in WT lungs, but largely preserved in Casp4/11^{-/-} infected lungs. Taken together, we conclude that CASP4/11 contributes to endothelial injury and instigation of the coagulation cascade during SARS-CoV-2 infection.

Given the tremendous effect reduction or depletion of CASP4 has on the symptoms, disease state, or severity of disease as shown herein, reduction of CASP4 and/or its downstream effectors are viable targets for the treatment, reduction, inhibition, decrease, and/or amelioration of a coronavirus infection, or the symptoms or disease state associated therewith. Accordingly, in one aspect, disclosed herein are methods of treating, inhibiting, reducing, decreasing, and/or ameliorating a coronaviral infection (such as, for example a SARS-CoV-2 (including, but not limited to the SARS-CoV-2 B1.351 variant, SARS-CoV-2B.1.1.7 (alpha), SARS-CoV-2B.1.1.7 variant mutant N501 Y (alpha), SARS-CoV-2 delta variant, SARS-CoV-2 P.1 variant, SARS-CoV-2 with T487K, P681R, and L452R mutations in B.1.617.2 (Delta), SARS-CoV-2 with K417N mutation in AY.1/AY.2 (Delta plus), SARS-CoV-2 with D614G, P681H, and D950N mutations in B.1.621 (Mu), SARS-CoV-2 with G75V, T76I, Δ246-252, L452Q, F490S, D614G, and T859N mutations in C.37 (Lambda), SARS-CoV-2 with T478K, Q498R, and H655Y mutations in B.1.1.529 (Omicron)) or the symptoms (thrombosis, pneumonia, inflammation (including, but not limited to inflammatory long damage), and tissue damage) or disease state (such as, for example, COVID-19) thereof comprising administering to a subject with a coronaviral infection an inhibitor of caspase 4 (CASP4)(such as, for example, an anti-CASP4 antibody, small molecule, protein, or an antisense oligonucleotide, an siRNA or shRNA that binds to and inhibits Casp4) or a neutrophil inhibitor (such as for example serpin B), or inhibitor of von Willebrand factor (vWF), IL-10, or Cxcl1. Inhibitors of CASP4, vWF, IL-1b, and Cxcl1 can include antibodies, small interfering RNA (siRNA), antisense oligonucleotide, short hairpin RNA (shRNA), proteins, and small molecules. For example, the antibody can be an anti-CASP4 antibody (such as, for example, 4B9 and/or MA5-26748), an anti-vWF antibody (including, but not limited to AJW200, ARC1779, Caplacizumab (ALX0081), 82D6A3, h6B4-Fab, GPG-290), an anti-IL10p antibody (canakinumab (ILARIS®), rilonacept (ARCALYST®), anakinra (KINERET), gevokizumab, and/or ly2189102), and/or an anti-Cxcl1 antibody (such as, for example, MA5-23745, MAB453-100, MM0208-9A18, and/or HL2401. Where an antisense oligonucleotide, siRNAs or shRNAs are used, the siRNA and/or shRNA can be obtained from commercially available sources such as origene, cellomicstech, Hindawi, thermofisher, vector biolabs, applied biological materials, and ABBEXA®. For example, an anti-Casp4 siRNA can be obtained from thermofisher, applied biological materials, origene; an anti-Casp4 shRNA can be obtained from vector biolabs, origene, ABBEXA®; an anti-Cxcl1 siRNA can be obtained from thermofisher or applied biological materials: an anti-Cxcl1 shRNA can be obtained from vector biolabs, origene, cellomicstech, ABBEXA®; an anti-i11b siRNA can be obtained from ABBEXA® or Hindawi; and an anti-i11b shRNA can be obtained from vector biolabs, origene, ABBEXA®.

It is understood and herein contemplated that the disclosed methods of treating, inhibiting, reducing, decreasing, and/or ameliorating a coronaviral infection are not limited to a particular coronaviral infection (such as, SARS-Cov-2), but can be performed on infected tissue samples from a subject infected with any coronavirus including but not limited to avian coronavirus (IBV), porcine coronavirus HKU15 (PorCoV HKU15), Porcine epidemic diarrhea virus (PEDV), HCoV-229E, HCoV-OC43, HCoV-HKU1, HCoV-NL63, SARS-CoV, SARS-CoV-2 (including, but not limited to the SARS-CoV-2 B1.351 variant, SARS-CoV-2B.1.1.7 (alpha), SARS-CoV-2B.1.1.7 variant mutant N501Y (alpha), SARS-CoV-2 delta variant, SARS-CoV-2 P.1 variant, SARS-CoV-2 with T487K, P681R, and L452R mutations in B. 1.617.2 (Delta), SARS-CoV-2 with K417N mutation in AY.1/AY.2 (Delta plus), SARS-CoV-2 with D614G, P681H, and D950N mutations in B.1.621 (Mu), SARS-CoV-2 with G75V, T76I, Δ246-252, L452Q, F490S, D614G, and T859N mutations in C.37 (Lambda), SARS-CoV-2 with T478K, Q498R, and H655Y mutations in B.1.1.529 (Omicron)), and MERS-CoV.

In one aspect, the subject being treated is not limited to a human subject, but can be a non-human primate, mouse, rat, pig, goat, cow, dog, cat, horse, sheep, or bird (including, but not limited to chicken, turkey, duck, quail, pigeon, or pheasant).

### 1. Nucleic Acids

There are a variety of molecules disclosed herein that are nucleic acid based, including for example various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantagous that the antisense molecule be made up of nucleotide analogs that reduce the degradation of the antisense molecule in the cellular environment.

Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules (small interfering RNA (siRNA), RNAi), short hairpin RNA (shRNA), aptamers, ribozymes, triplex forming molecules, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA of any of the disclosed nucleic acids, such as Casp4, i1-1b, cxcl1, and vWF. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (k_{d}) less than or equal to 10⁻⁶, 10⁻⁸, 10⁻¹⁰, or 10⁻¹². A representative sample of methods and techniques which aid in the design and use of antisense molecules can be found in the following non-limiting list of U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with k_{d}s from the target molecule of less than 10⁻¹² M. It is preferred that the aptamers bind the target molecule with a k_{d} less than 10⁻⁶, 10⁻⁸, 10⁻¹⁰, or 10⁻¹². Aptamers can bind the target molecule with a very high degree of specificity. For example, aptamers have been isolated that have greater than a 10000 fold difference in binding affinities between the target molecule and another molecule that differ at only a single position on the molecule (U.S. Pat. No. 5,543,293). It is preferred that the aptamer have a k_{d} with the target molecule at least 10, 100, 1000, 10,000, or 100,000 fold lower than the k_{d} with a background binding molecule. It is preferred when doing the comparison for a polypeptide for example, that the background molecule be a different polypeptide. Representative examples of how to make and use aptamers to bind a variety of different target molecules can be found in the following non-limiting list of U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes, (for example, but not limited to the following U.S. Pat. Nos. 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203, WO 9858058 by Ludwig and Sproat, WO 9858057 by Ludwig and Sproat, and WO 9718312 by Ludwig and Sproat) hairpin ribozymes (for example, but not limited to the following U.S. Pat. Nos. 5,631,115, 5,646,031, 5,683,902, 5,712,384, 5,856,188, 5,866,701, 5,869,339, and 6,022,962), and tetrahymena ribozymes (for example, but not limited to the following U.S. Pat. Nos. 5,595,873 and 5,652,107). There are also a number of ribozymes that are not found in natural systems, but which have been engineered to catalyze specific reactions de novo (for example, but not limited to the following U.S. Pat. Nos. 5,580,967, 5,688,670, 5,807,718, and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in the following non-limiting list of U.S. Pat. Nos. 5,646,042, 5,693,535, 5,731,295, 5,811,300, 5,837,855, 5,869,253, 5,877,021, 5,877,022, 5,972,699, 5,972,704, 5,989,906, and 6,017,756.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. It is preferred that the triplex forming molecules bind the target molecule with a k_{d} less than 10⁻⁶, 10⁻⁸, 10⁻¹⁰, or 10⁻¹². Representative examples of how to make and use triplex forming molecules to bind a variety of different target molecules can be found in the following non-limiting list of U.S. Pat. Nos. 5,176,996, 5,645,985, 5,650,316, 5,683,874, 5,693,773, 5,834,185, 5,869,246, 5,874,566, and 5,962,426.

External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)).

Similarly, eukaryotic EGS/RNAse P-directed cleavage of RNA can be utilized to cleave desired targets within eukarotic cells. (Yuan et al., Proc. Natl. Acad. Sci. USA 89:8006-8010 (1992); WO 93/22434 by Yale; WO 95/24489 by Yale; Yuan and Altman, EMBO J 14:159-168 (1995), and Carrara et al., Proc. Natl. Acad. Sci. (USA) 92:2627-2631 (1995)). Representative examples of how to make and use EGS molecules to facilitate cleavage of a variety of different target molecules be found in the following non-limiting list of U.S. Pat. Nos. 5,168,053, 5,624,824, 5,683,873, 5,728,521, 5,869,248, and 5,877,162.

### 2. Antibodies

### (1) Antibodies Generally

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with CASP4, vWF, IL1β, or Cxcl1. The antibodies can be tested for their desired activity using the in vitro assays described herein, or by analogous methods, after which their in vivo therapeutic and/or prophylactic activities are tested according to known clinical testing methods. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity.

The disclosed monoclonal antibodies can be made using any procedure which produces mono clonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

The monoclonal antibodies may also be made by recombinant DNA methods. DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

As used herein, the term “antibody or fragments thereof” encompasses chimeric antibodies and hybrid antibodies, with dual or multiple antigen or epitope specificities, and fragments, such as F(ab′)2, Fab′, Fab, Fv, sFv, scFv, and the like, including hybrid fragments. Thus, fragments of the antibodies that retain the ability to bind their specific antigens are provided. For example, fragments of antibodies which maintain CASP4, vWF, IL-1b, or Cxcl1 binding activity are included within the meaning of the term “antibody or fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or fragments thereof” are conjugates of antibody fragments and antigen binding proteins (single chain antibodies).

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

### (2) Human Antibodies

The disclosed human antibodies can be prepared using any technique. The disclosed human antibodies can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551-255 (1993); Jakobovits et al., Nature, 362:255-258 (1993); Bruggermann et al., Year in Immunol., 7:33 (1993)). Specifically, the homozygous deletion of the antibody heavy chain joining region (J(H)) gene in these chimeric and germ-line mutant mice results in complete inhibition of endogenous antibody production, and the successful transfer of the human germ-line antibody gene array into such germ-line mutant mice results in the production of human antibodies upon antigen challenge. Antibodies having the desired activity are selected using Env-CD4-co-receptor complexes as described herein.

### (3) Humanized Antibodies

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an sFv, Fv, Fab, Fab′, F(ab′)2, or other antigen-binding portion of an antibody) which contains a portion of an antigen binding site from a non-human (donor) antibody integrated into the framework of a human (recipient) antibody.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525 (1986), Reichmann et al., Nature, 332:323-327 (1988), and Presta, Curr. Opin. Struct. Biol., 2:593-596 (1992)).

Methods for humanizing non-human antibodies are well known in the art. For example, humanized antibodies can be generated according to the methods of Winter and co-workers (Jones et al., Nature, 321:522-525 (1986), Riechmann et al., Nature, 332:323-327 (1988), Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

### (4) Administration of Antibodies

Administration of the antibodies can be done as disclosed herein. Nucleic acid approaches for antibody delivery also exist. The broadly neutralizing anti-CASP4, anti-IL-1b, anti-Cxcl1, or anti-vWF antibodies and antibody fragments can also be administered to patients or subjects as a nucleic acid preparation (e.g., DNA or RNA) that encodes the antibody or antibody fragment, such that the patient's or subject's own cells take up the nucleic acid and produce and secrete the encoded antibody or antibody fragment. The delivery of the nucleic acid can be by any means, as disclosed herein, for example.

### 3. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399409 (1991)).

### a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, P A 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

### C. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1: Caspase-4/11 Exacerbates Disease Severity in SARS-CoV-2 Infection by Promoting Inflammation and Thrombosis

### a) Results

(1) CASP4/11 Expression is Elevated in Lungs during SARS-CoV-2 Infections of Mice and Humans and Correlates with Disease Severity in Humans.

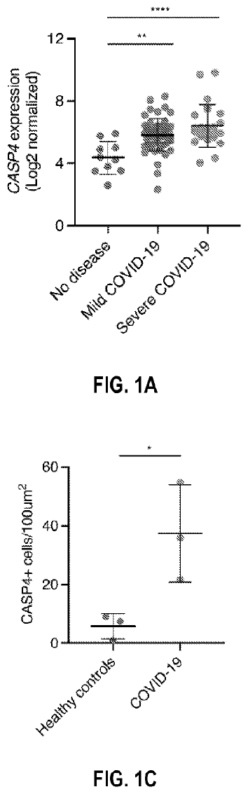

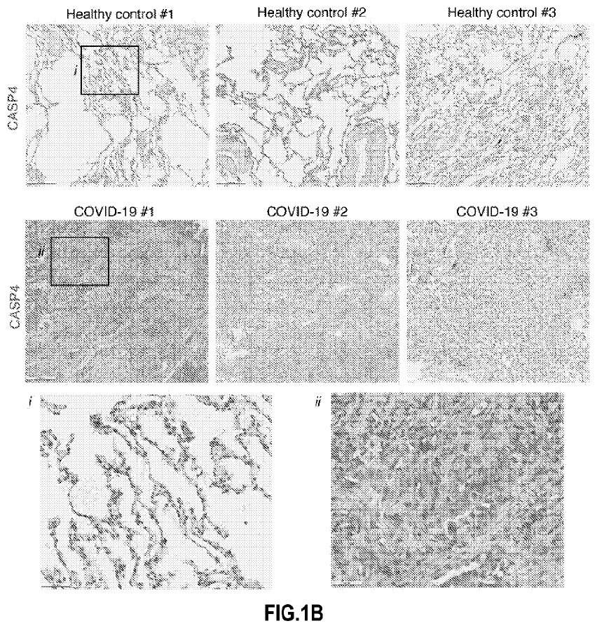

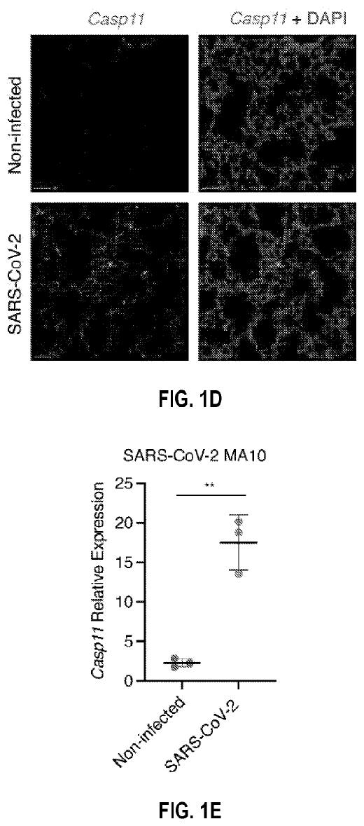

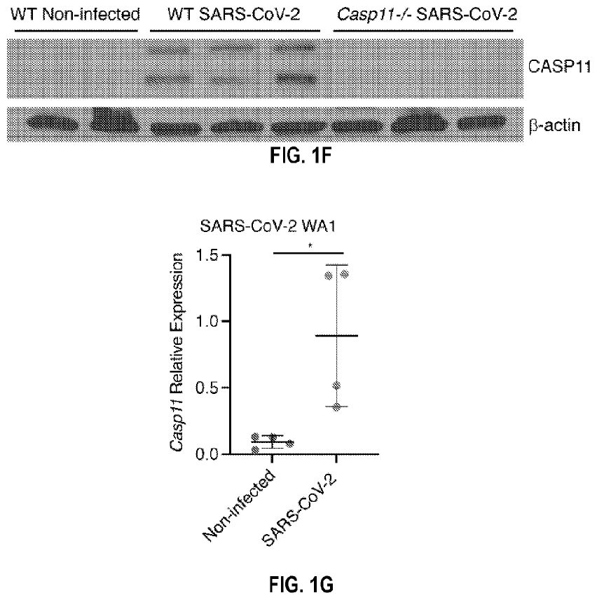

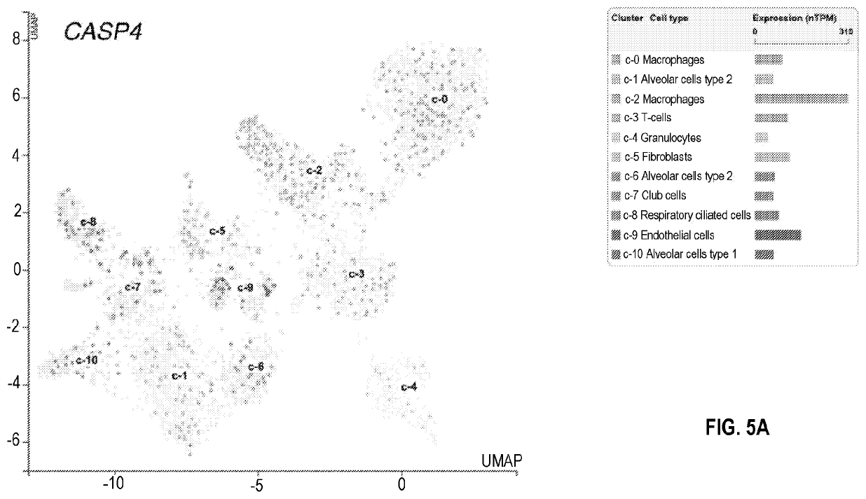

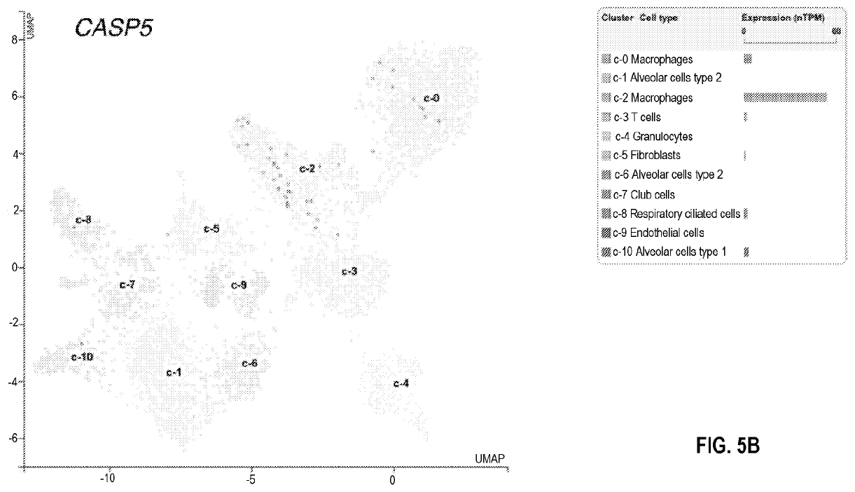

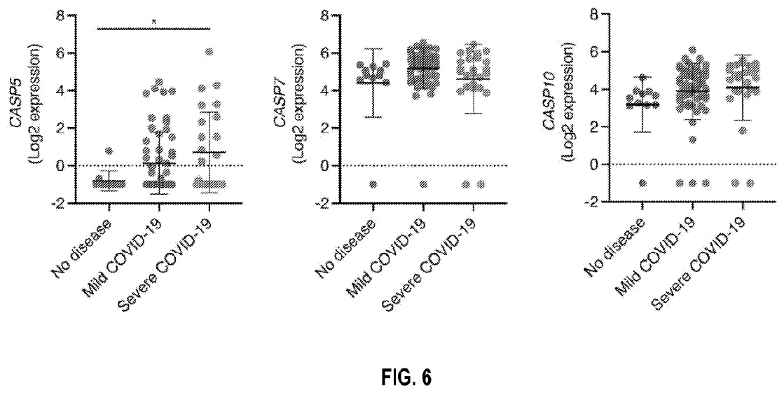

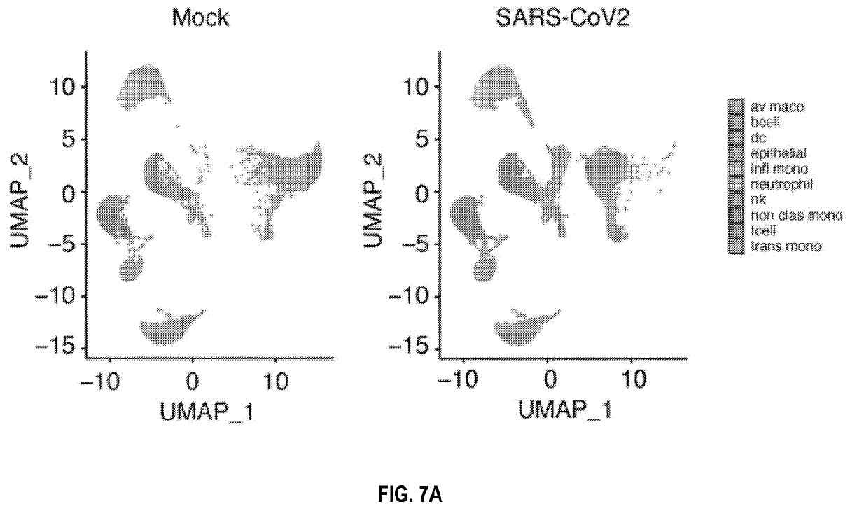

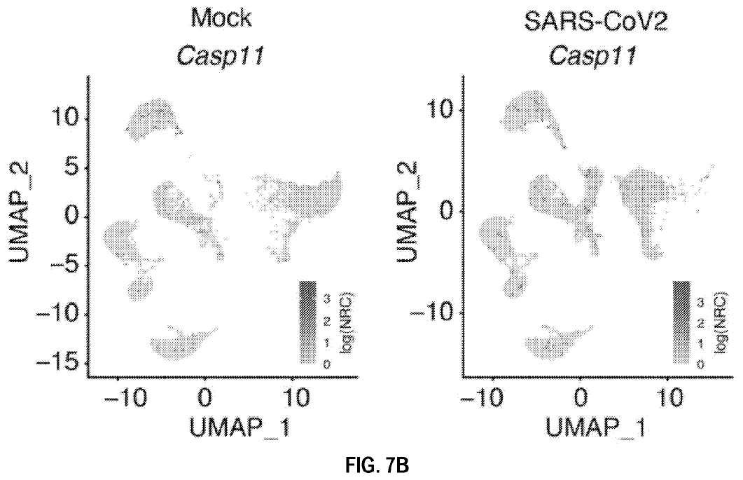

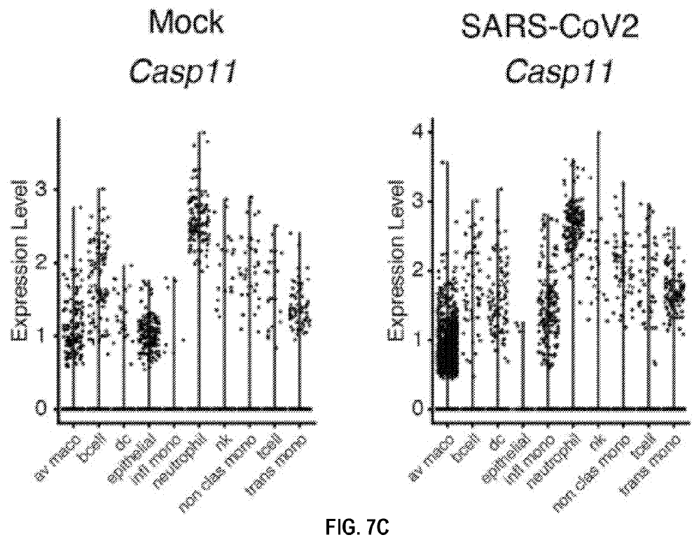

CASP4/11 is reported to be widely expressed in epithelial and endothelial cells, where it is involved in the response of these cells to bacterial lypopolysacchride (LPS). CASP4/11 also has well-characterized roles in responding to gram-negative and -positive bacteria in immune cells, including macrophages and neutrophils. To gain a better understanding of CASP4 expression patterns, we examined human lung single-cell RNA sequencing (scRNAseq) data for CASP4 expression. These data show CASP4 transcripts in all lung cell types including type I and type H pneumocytes, ciliated cells, and club cells and indicated particularly prominent expression in macrophages and endothelial cells (FIG. 5A). Expression of the related protein CASP5 was restricted to macrophages (FIG. 5B). While CASP4/11 can be expressed in resting cells, it is highly induced in response to bacterial infections. The analysis of publicly available RNA sequencing data of nasopharyngeal swab material from subjects with SARS-CoV-2 and healthy donors (Gene Expression Omnibus [GEO] accession No. GSE163151) revealed that CASP4 is highly expressed in the airway of SARS-CoV-2-infected patients and that expression levels increase with disease severity (FIG. 1A). CASP5 expression was also up-regulated in infected samples, while CASP7 and CASP10 expression served as controls that were unaffected by infection status (FIG. 6). Additionally, we found that human lung sections from COVID-19 patients show higher levels of CASP4 staining compared with healthy lung controls (FIG. 1B), owing to greater numbers of CASP4-positive cells in the infected lung tissue (FIG. 1C). We then performed intranasal infection of C57BL/6 wild-type (WT) mice with pathogenic mouse-adapted SARS-CoV-2 (strain MA10) and found that infection strongly induces Casp11 expression throughout murine lung tissue within 4 d of infection, as detected by RNAscope in situ hybridization (ISH) (FIG. 1D) and confirmed by qRT-PCR (FIG. 1E). The level of CASP11 protein likewise went from low to highly expressed in response to SARS-CoV-2 infection of murine lungs (FIG. 1F). We further examined infection of K18-hACE2 mice expressing the human ACE2 receptor using human isolate SARS-CoV-2 strain USA-WA1/2020 (W A1). Similar to mouse-adapted SARS-CoV-2, the nonadapted human virus strongly induced the lung expression of CASP11 as demonstrated by qRT-PCR (FIG. 1G). These results are additionally supported by analysis of scRNAseq of mouse lung cells, which similarly identified wide expression of CASP11 that is sustained or enhanced in most cell types following SARS-CoV-2 infection, including high CASP4/11 expression in inflammatory cells recruited to the lung following infection (FIG. 7). Overall, CASP4 is highly expressed in the lungs of COVID-19 patients, and CASP11 is similarly increased upon SARS-CoV-2 infection of mice.

### (2) Casp11 Deficiency Reduces Disease Severity in SARS-CoV-2-Infected Mice.

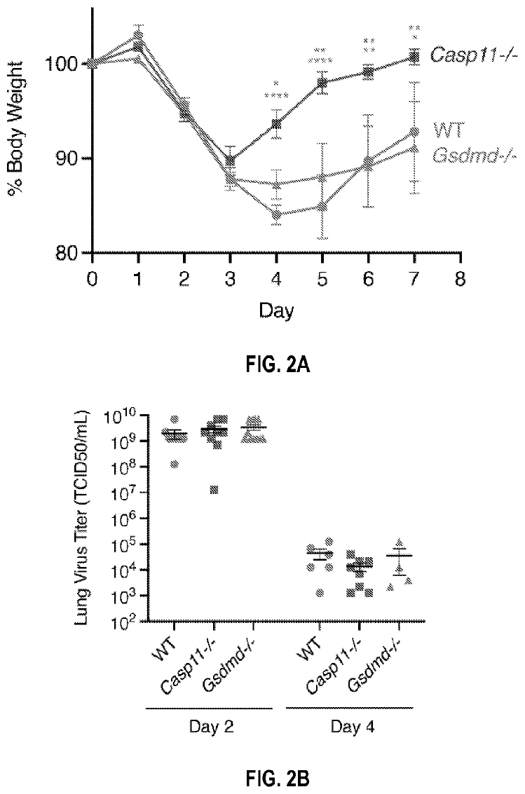

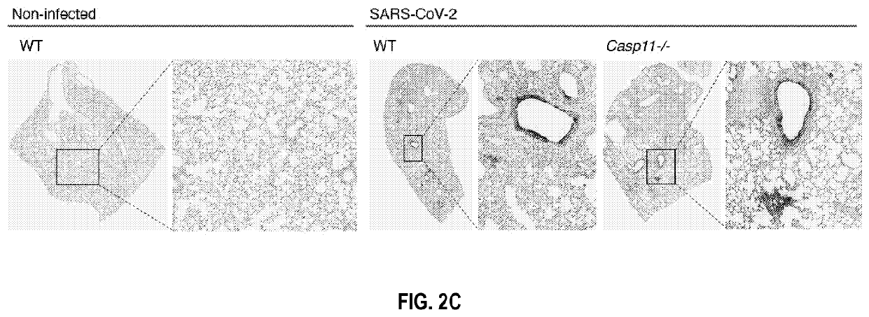

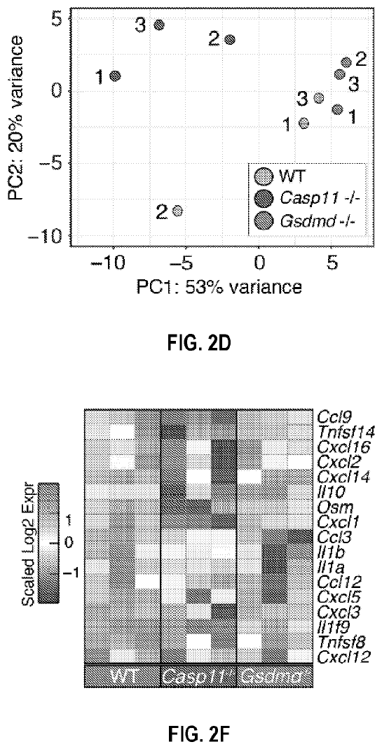

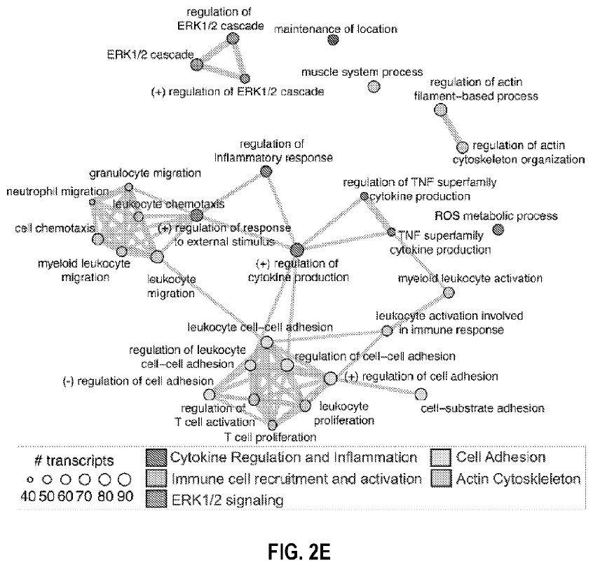

We next examined whether CASP4/11 regulates disease severity caused by SARS-CoV-2 infection. WT, Casp11^{-/-}, and Gsdmd^{-/-} mice were infected with SARS-CoV-2 MA10 for comparison of weight loss, a commonly used indicator of overall infection severity in mice. We found that WT mice lost a significant percentage of their body weight between days 1 and 4 after infection, followed by partial recovery of weight up to day 7, at which point we ended our experiments (FIG. 2A). Casp11^{-/-} mice, on the other hand, lost weight only up to day 3 and then rapidly recovered fully to their original weight by day 5 (FIG. 2A). In comparison, weight loss of Gsdmd^{-/-} mice was not significantly different from that of WT mice (FIG. 2A). These data indicate that CASP4/11 promotes disease severity during SARS-CoV-2 infection and that this function is not mediated by GSDMD.

To determine whether differences in disease severity could be explained by differences in viral replication, we quantified live virus titers in WT, Casp11^{-/-}, and Gsdmd^{-/-} mouse lungs at 2 and 4 d after infection. We found that viral loads were similar with no statistical difference between the groups at either time point (FIG. 2B). We also observed, in agreement with previous reports, that viral titers were decreased at day 4 compared with day 2 in all groups, demonstrating that neither CASP7/11 nor GSDMD is required for viral clearance mechanisms in mice (FIG. 2B). To corroborate these findings, lung sections from WT and Casp11^{-/-} mice were stained for SARS-CoV-2 nucleocapsid protein, and similar staining patterns were observed with prominent infection of cells lining the airways and neighboring alveoli (FIG. 2C). Overall, these results demonstrate that loss of CASP4/11, but not GSDMD, prevents severe disease in SARS-CoV-2 infection without affecting virus replication or clearance.

### (3) Casp11^{-/-} Controls Specific Inflammatory Gene Signatures in SARS-CoV-2-Infected Lungs.

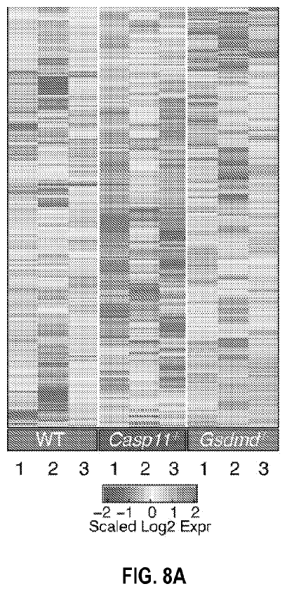

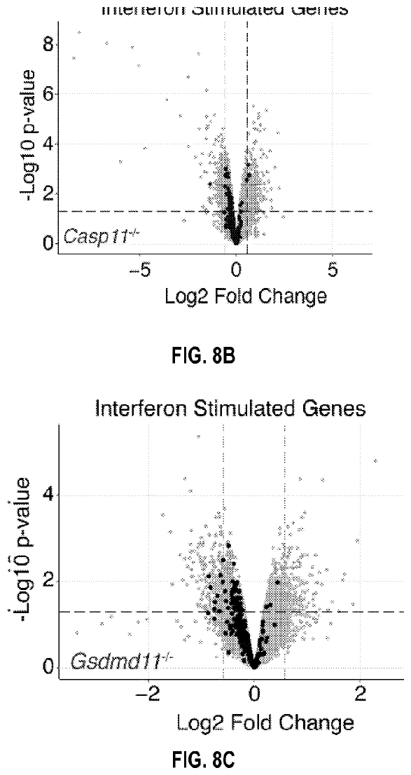

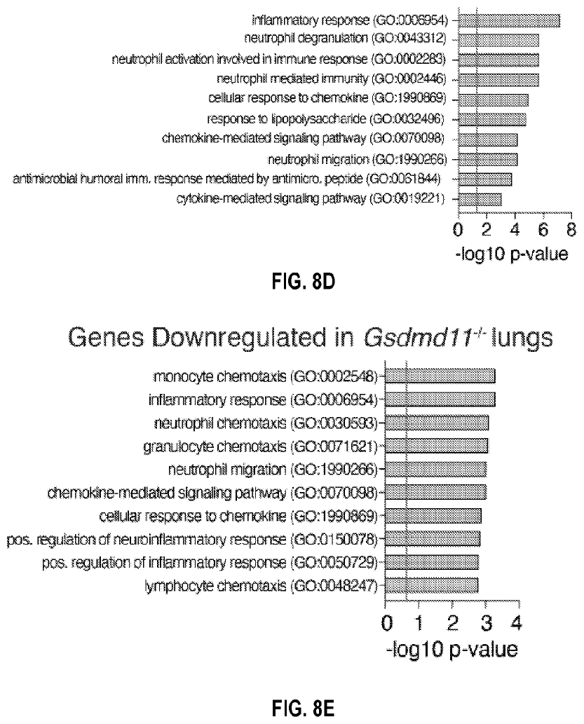

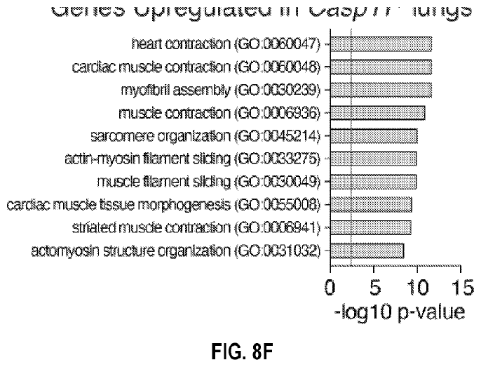

To examine global transcriptional effects of CASP4/11 and GSDMD in the lung during SARS-CoV-2 infections, we infected WT, Casp11^{-/-}, and Gsdmd^{-/-} mice and performed RNA sequencing on lung RNA at 2 d after infection. Day 2 was chosen because it is the peak of virus replication in the lungs of mice. Distinct gene signatures in WT, Casp11^{-/-}, and Gsdmd^{-/-} infected lungs were seen using dimensionality reduction approaches, with the Casp11^{-/-} infected lung profiles showing the most extensive divergence in gene expression patterns (FIG. 2D). We contrasted the significant gene expression changes (P value<0.05) in infected Casp11^{-/-} and Gsdmd lungs relative to WT mice to understand how these genes impact the transcriptional landscape in terms of differentially expressed genes (either significantly up-regulated or down-regulated) (FIG. 8A). Functional analysis of differentially expressed genes in Casp11^{-/-} versus WT lungs revealed an enrichment for genes corresponding to immunological pathways involved in cytokine production and inflammation (red), immune cell migration and activation (orange), cell adhesion (pink), and extracellular signal-related kinase (ERK)1/2 signaling (green) (FIG. 2E). In accordance with known actin polymerization regulation imparted by CASP11, the absence of Casp11 in SARS-CoV-2 infection also resulted in expression changes in genes involved in actin regulatory pathways (blue) (FIG. 2E). Since sensing of virus replication by cells generally induces IFN-mediated antiviral responses, we investigated whether CASP11 or GSDMD shape the antiviral gene program during SARS-CoV-2 infection. First, we specifically examined the expression of IFN-stimulated genes (ISGs), which are abundantly up-regulated by type I IFN stimulation in murine airway epithelial cells. Deficiency of CASP11 or GSDMD did not result in differential ISG expression relative to WT infected lungs (log 2 fold change [LFC]|0.58|; P value<0.05) expression relative to WT infected lungs (FIGS. 8B and 8C). This lack of interplay between CASP11 and GSDMD with the type I IFN antiviral pathway is consistent with the similar viral titers observed in our distinct animal groups (FIG. 2B).

Specific examination of cytokine and chemokine genes revealed a statistically significant down-regulation of several important inflammatory mediators in the absence of Casp11, including cytokines IL1β, IL1α, and IL1f9 and chemokines Cxcl1, Cxcl2, Cxcl14, Cxcl3, Cxcl5, and Ccl3 (FIG. 2F). These findings are consistent with ERK activation downstream of CXCL1 and CXCL3 signaling as highlighted in FIG. 2E. Knockout of Gsdmd had less impact on the magnitude of cytokine and chemokine expression compared to Casp11 knockout (FIG. 2F). Overall, our results demonstrate that CASP11 controls a specific subset of inflammatory responses during SARS-CoV-2 infection.

### (4) CASP11 Promotes the Production of Specific Inflammatory Mediators in Response to SARS-CoV-2 In Vivo and In Vitro.

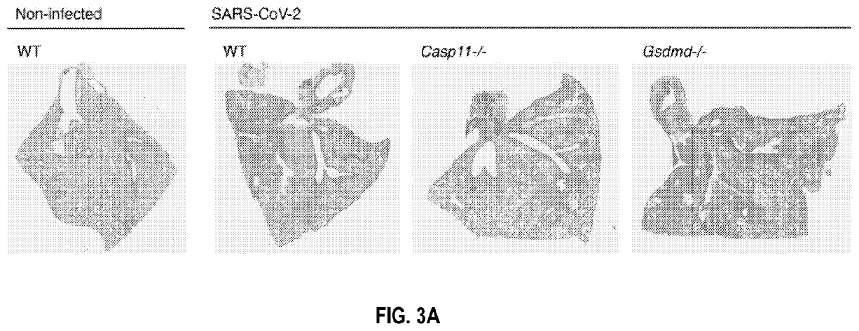

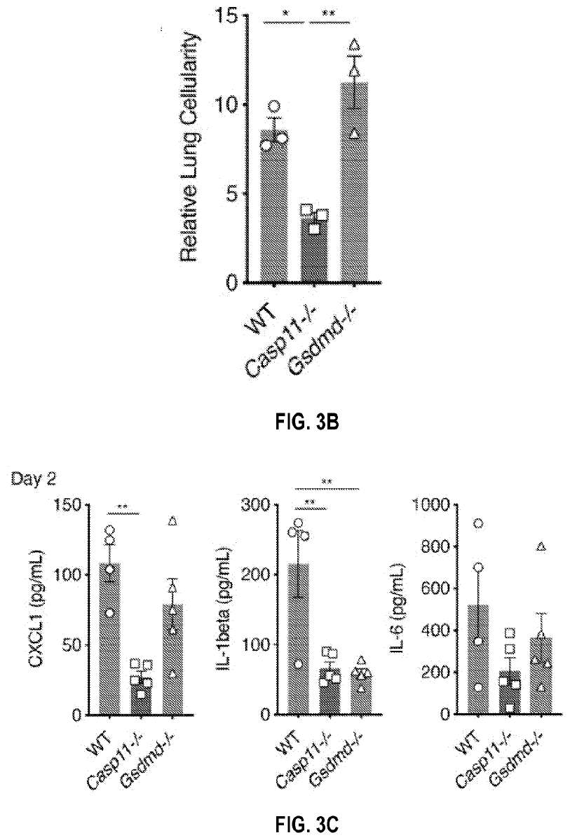

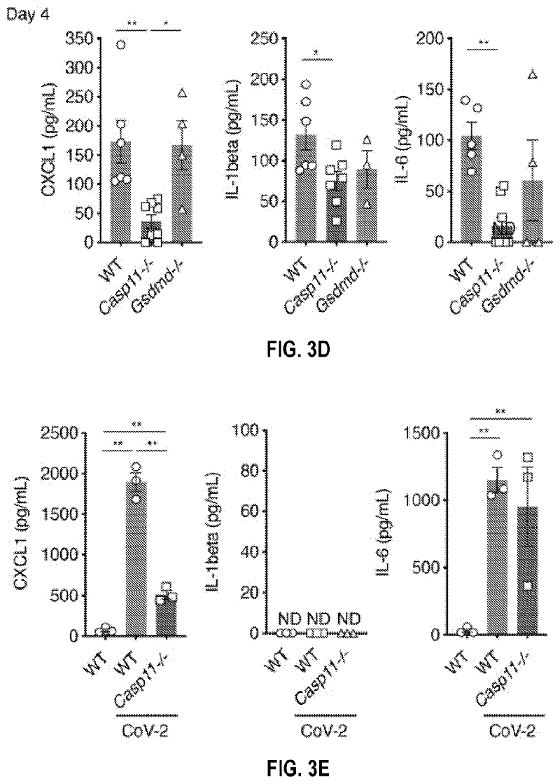

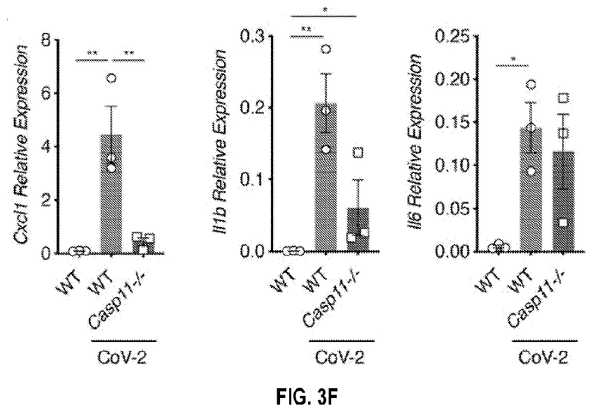

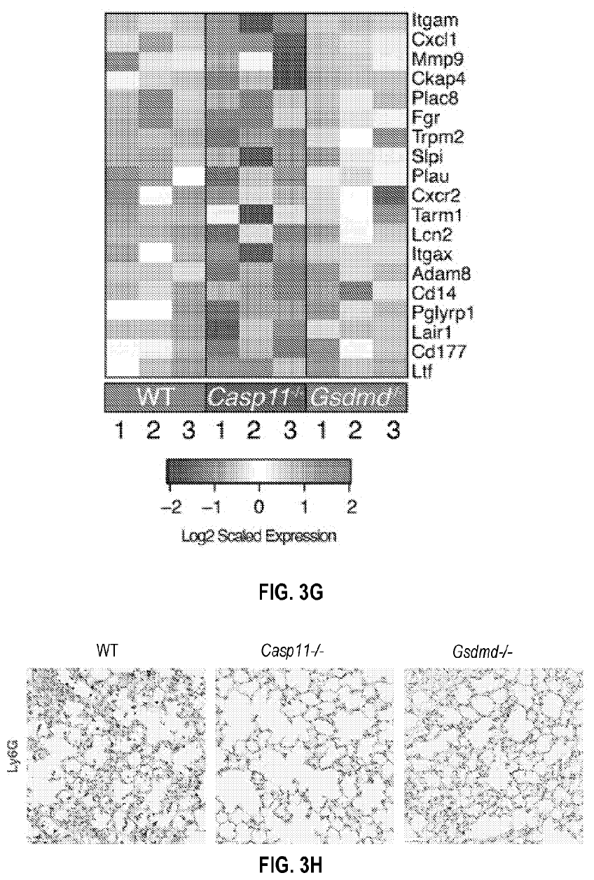

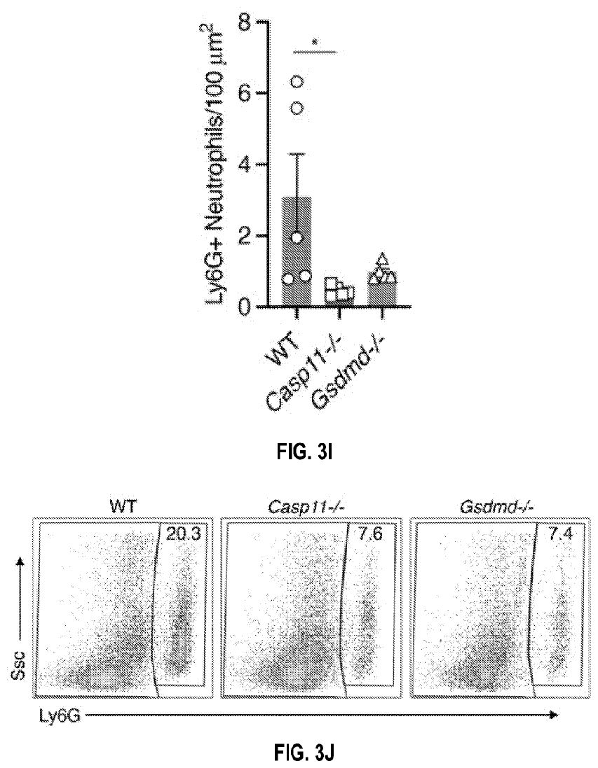

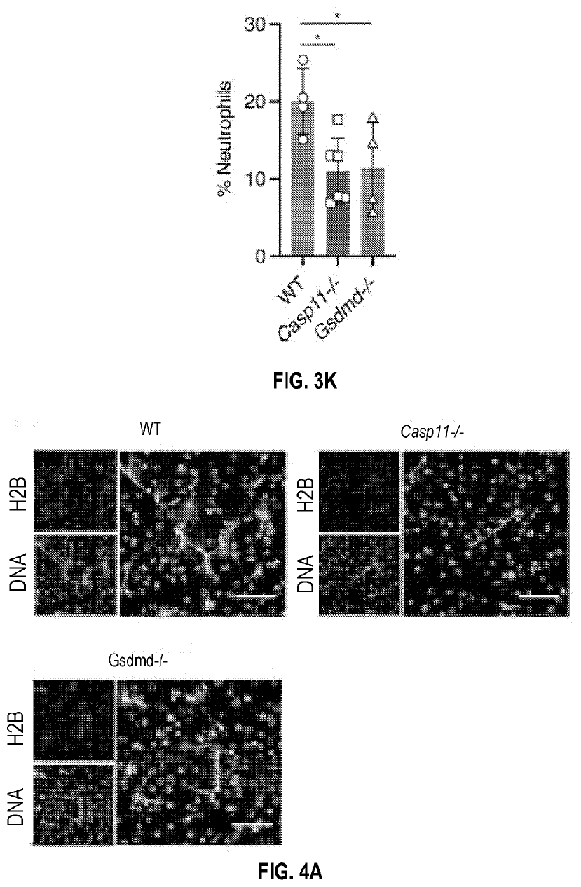

To examine the role of CASP11 in mediating the pathological hallmarks of SARS-CoV-2 pulmonary infection, lung sections from infected WT, Casp11^{-/-}, and Gsdmd^{-/-} mice were fixed and stained with hematoxylin and eosin (H&E). Sections from all infected animals showed areas of consolidated lung tissue indicative of cellular infiltration and inflammation that was absent in noninfected control tissue (FIG. 3A). However, WT and Gsdmd^{-/-} lung sections showed more severe tissue consolidation and cell infiltration throughout a greater portion of the lung than that seen in Casp11^{-/-} mice. We thus quantified cell area versus airway space to determine cellularity scores indicative of pathology for tissue sections from individual mice. We observed significantly decreased SARS-CoV-2-induced lung pathology in Casp11^{-/-} mice compared to WT and Gsdmd^{-/-} mice (FIGS. 3A and 3B), correlating with the preservation of Casp11^{-/-} body weight and their faster recovery (FIG. 2A).

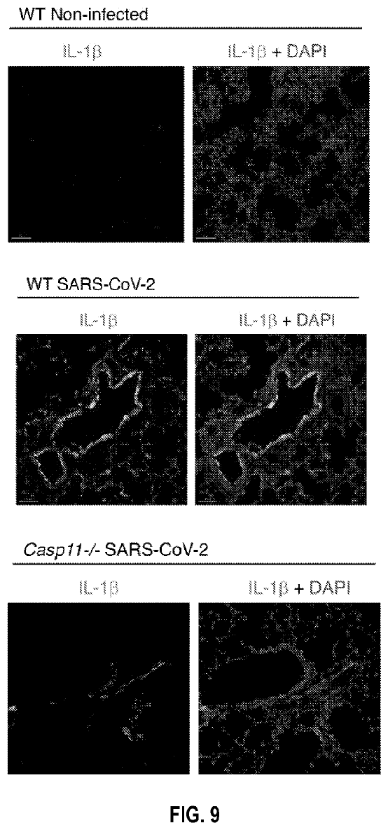

Guided by our transcriptomic results indicating that a critical subset of inflammatory mediators are controlled by CASP11 (FIG. 2F), we measured levels of CXCL1, IL-1β, and IL-6 by enzyme-linked immunosorbent assay (ELISA) in lung homogenates from infected animals at 2 and 4 d after infection (FIGS. 3C and 3D). IL-1β was lower in the lungs of both Casp11^{-/-} and Gsdmd^{-/-} mice at 2 d after infection when compared to WT (FIG. 3C). Moreover, IL-1β staining in lung tissue sections revealed more IL-1β in WT than that observed in Casp11^{-/-} mice (FIG. 9). Similarly, the production of CXCL1 was dependent on Casp/at both time points, though this was independent of Gsdmd (FIGS. 3C and 3D). Average levels of IL-6 were partially decreased in Casp11^{-/-} lungs, with a statistically significant difference between WT and Casp11^{-/-} lungs at day 4 (FIGS. 3C and 3D). These results corroborate and expand our day 2 transcriptomic analysis in which expression of IL1β and Cxcl1 was decreased (FIG. 2F) and demonstrate that production of a critical subset of inflammatory mediators in the lung is dependent on CASP11^{-/-} during SARS-CoV-2 infection.

To determine the role of CASP11^{-/-} in the response of lung macrophages to SARS-CoV-2, we purified mature primary macrophages from lungs of WT and (Casp11^{-/-} mice and infected them with SARS-CoV-2 MA10 Culture supernatants and cellular RNA were collected and measured for IL-1β, IL-6, and CXCL1 protein and transcript levels, respectively. Compared with noninfected cells. CXCL1 protein and RNA transcripts were detected at high levels upon infection of WT macrophages but were poorly produced by Casp11^{-/-} cells (FIGS. 3E and 3F). Interestingly, IL-1β transcripts were also induced in a CASP11-dependent manner, but secreted protein was not detected in either group (FIGS. 3E and 3F). Distinctly, protein and transcript levels of IL-6 did not significantly differ between WT and Casp11^{-/-} cells (FIGS. 3E and 3F). These results confirm our in vivo measurements and further demonstrate that CASP4/11 is an important cellular regulator of specific cytokines and chemokines, including CXCL1 and IL-1β, in response to SARS-CoV-2.

(5) CASP11 Promotes Lung Neutrophil Responses during SARS-CoV-2 Infection.

To better understand the biological processes regulated by Casp11, we further analyzed the functional gene enrichment categories of the 236 genes most down-regulated (LFC less than −0.58; P value<0.05) in (Casp11^{-/-} lungs. A striking enrichment of neutrophil-related gene signatures emerged that included neutrophil-specific markers (e.g., Cd177 and Cxcr2), neutrophil degranulation genes (e.g., Pglryp1, Ckap4, Adam8, and Plac8), and neutrophil complement receptors (Itgam and Itgax), among others (FIG. 3G and FIG. 8D). Additionally, genes associated with the response to tissue damage from neutrophils (Slpi and Lair1) were also decreased in the absence of Casp11 relative to WT lungs (FIG. 3G). These results are consistent with decreased gene expression for the neutrophil chemoattractant CXCL1 (FIGS. 3C and 3D), as well as with previous reports of neutrophil regulation by CASP4/11 through effects on actin.

Notably, expression of these neutrophil signature genes in Gsdmd^{-/-} lungs was less affected than in Casp11^{-/-} lungs (FIG. 3G), though other genes that are down-regulated in the absence of Gsdmd were associated with dysregulation of other immune pathways (FIG. 8E). Conversely, analysis of genes up-regulated in the absence of Casp11 revealed a putative association with muscle-specific pathways (FIG. 8F), while genes most up-regulated in Gsdmd^{-/-} lungs were not enriched for any specific functional pathways. Overall, these analyses most prominently demonstrate that CASP4/11 is required for robust production of specific inflammatory mediators as well as neutrophil recruitment and functions in the lung during SARS-CoV-2 infection.

To further examine the role of neutrophils in SARS-CoV-2 infection, lung sections from WT, Casp11^{-/-}, and GsdmD^{-/-} mice were stained for the neutrophil marker Ly6G (FIG. 3H). Quantification of Ly6G-positive cells demonstrated fewer neutrophils in Casp11^{-/-} and GsdmD^{-/-} lung sections when compared to WT, with a statistically significant difference seen when comparing WT and Casp11^{-/-}, but not between Casp11^{-/-} and GsdmD^{-/-}, mice (FIG. 3I). These findings were corroborated by flow cytometric analysis quantifying the percentage of Ly6G^{high} neutrophils among the CD45⁺ immune cells in lung single-cell suspensions from SARS-CoV-2-infected WT, Casp11^{-/-}, and GsdmD^{-/-} mice (FIGS. 3J and 3K).

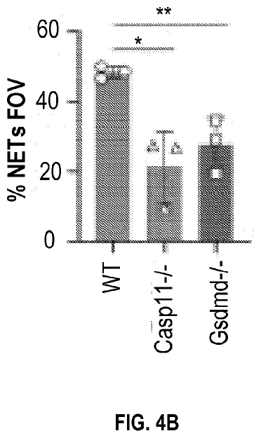

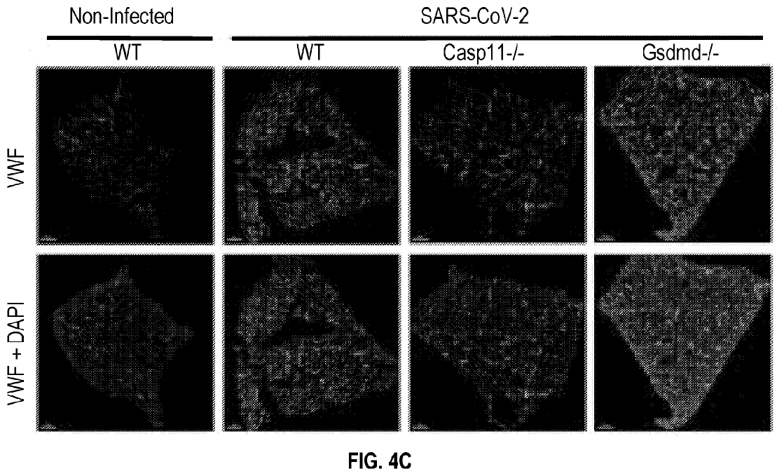

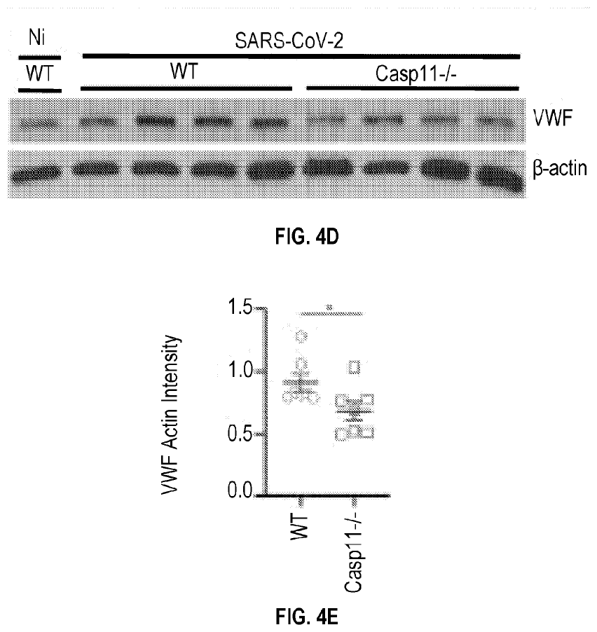

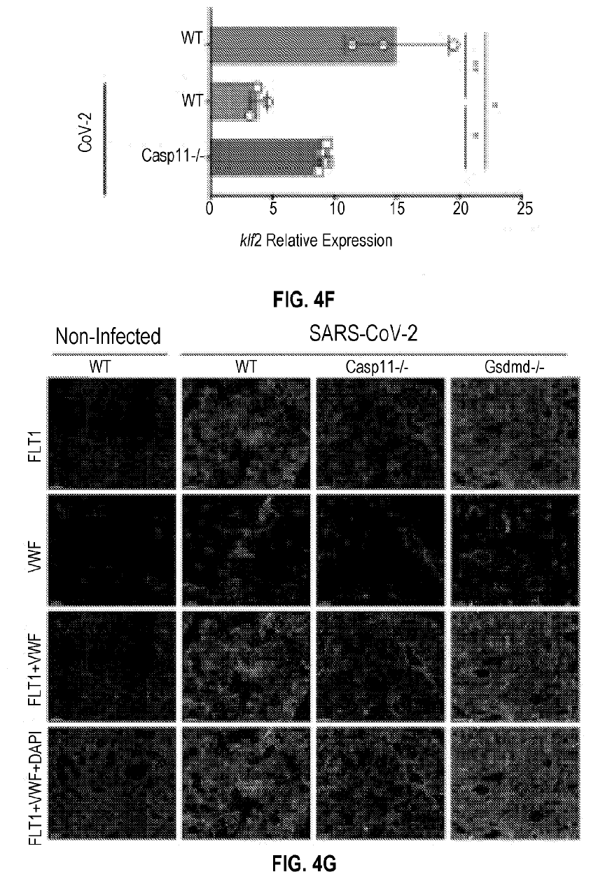

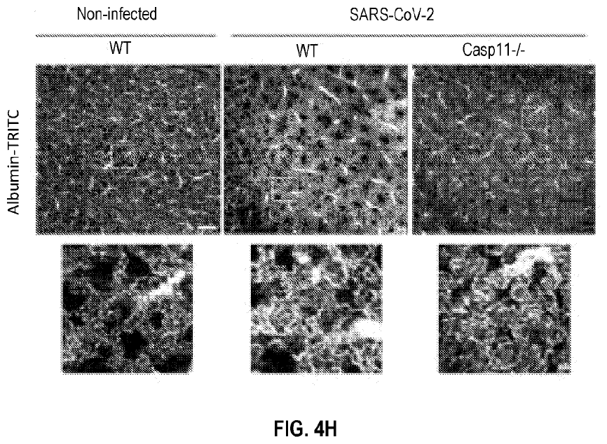

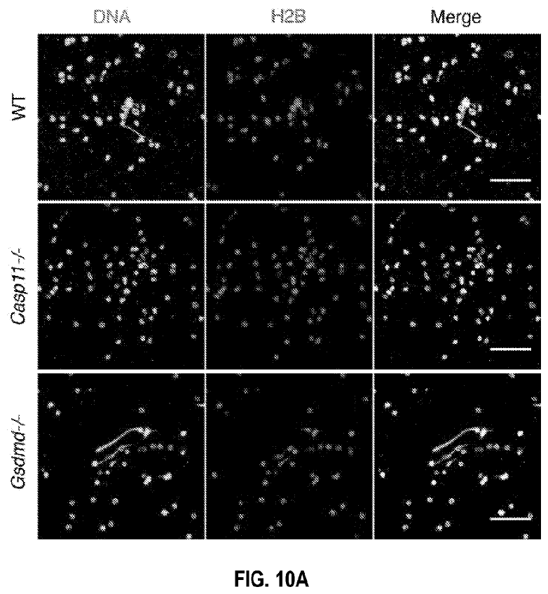

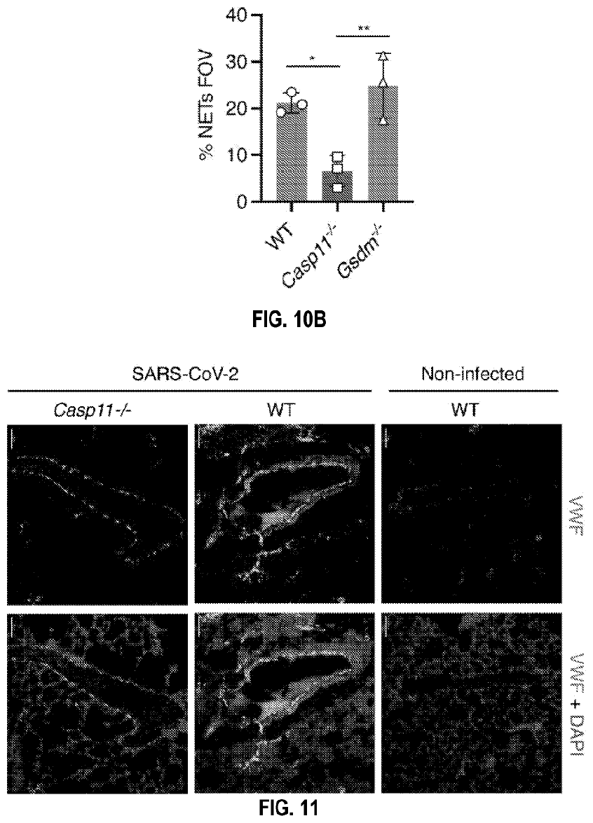

One of the main neutrophil-mediated functions is formation of neutrophil extracellular traps (NETs), which contain released chromatin that can immobilize pathogens and trigger immunothrombosis, especially during SARS-CoV-2 infection through platelet-neutrophil interactions. We were unable to detect direct infections of neutrophils with SARS-CoV-2. Thus, to determine if CASP11 and GSDMD modulate neutrophil functions during SARS-CoV-2 infection, WT, Casp11^{-/-}, and Gsdmd^{-/-} neutrophils were treated with phorbol myristate acetate (PMA) (FIGS. 4A and 4B) or culture supernatants from WT epithelial cells infected with SARS-CoV-2 to simulate the virus-induced inflammatory milieu (FIG. 10). Casp11^{-/-} neutrophils were largely defective in forming NETs in response to all conditions compared to WT neutrophils, which formed NETs in response to all conditions (FIGS. 4A and 4B and FIG. 10). Together, our data demonstrate that lungs of SARS-CoV-2-infected Casp11 mice contain fewer neutrophils than infected WT lungs, and Casp11^{-/-} neutrophils largely fail to undergo NETosis.

### (6) The Lack of CASP11 Reduces VWF Levels and Increases Vascular Integrity in Response to SARS-CoV-2.

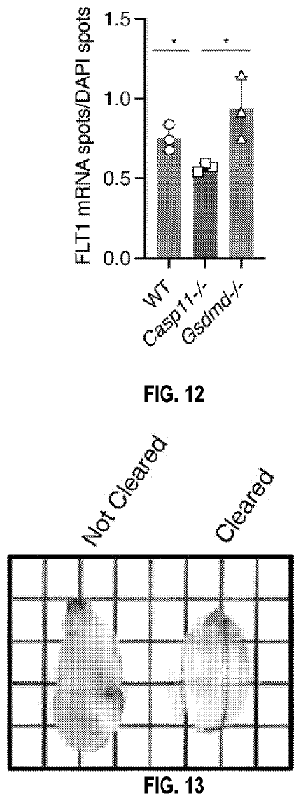

SARS-CoV-2 infection is accompanied by long-term sequelae mediated in part by vascular damage and thrombosis. Given that we noted decreased neutrophil gene signatures in Casp11^{-/-} lungs upon infection and since tissue infiltration by neutrophils can activate blood clotting cascades and thrombosis, we examined whether the production of von Willebrand factor (vWF), a marker for endothelial damage, which is essential to thrombus formation, is regulated by CASP4/11. Using RNAscope ISH technology, we found significantly more blood vessels expressing VWF messenger RNA (mRNA) in the lung vascular architecture of SARS-CoV-2-infected WT mice when compared to Casp11^{-/-} lungs at day 4 after infection (FIG. 4C and FIG. 11). Immunoblot analysis of lung homogenates confirmed that VWF was significantly lower in lungs of Casp11^{-/-} mice when compared to WT lungs (FIGS. 4D and 4E). Notably, lung sections from SARS-CoV-2-infected Gsdmd^{-/-} mice showed more staining for VWF than Casp11^{-/-} mice (FIG. 4C). We thus conclude that CASP4/11 is required for the accumulation of VWF in the lungs during SARS-CoV-2 infection. Furthermore, we examined the expression of Kruppel-Like Factor 2 (KLF2) in WT and Casp11^{-/-} SARS-CoV-2-infected lungs since KLF2 is an endothelial cell protective transcription factor that exerts anti-inflammatory and antithrombotic functions and maintains the integrity of the vasculature. We found that KLF2 expression was significantly reduced after SARS-CoV-2 infection in WT lungs but was largely preserved in Casp1^{-/-} infected lungs (FIG. 4F). To confirm the source of VWF, lung sections from Casp11^{-/-} and Gsdmd^{-/-} mice were processed for the simultaneous detection of endothelial marker vascular endothelial growth factor (VEGF) receptor 1 (FLT1) and VWF mRNA. We found that VWF RNA colocalized with FLT1, which was also up-regulated in WT and Gsdmd^{-/-} but not Casp11^{-/-} lung sections (FIG. 4G and FIG. 12). Moreover, we examined the vascular architecture in the cleared lungs of SARS-CoV-2-infected mice by using fluorophore-conjugated albumin and tissue clearing (FIG. 13). The vascular tracing revealed distinctive vascular features in WT SARS-CoV-2-infected lungs with pronounced vascular thickening, obliteration, and angiogenesis/neovascularization (FIG. 4H). In stark contrast, Casp11^{-/-} infected lung vasculature did not show these abnormalities, indicating less endothelial damage/dysfunction (FIG. 4H). Taken together, these findings indicate that CASP4/11 contributes to instigation of the coagulation cascade and induction of vascular changes in the lung during SARS-CoV-2 infection.

### b) Discussion

The medical and research communities have met challenges in identifying specific inflammatory mediators that can be targeted to ameliorate SARS-CoV-2 pathogenesis without impairing beneficial aspects of the immune response, such as viral clearance. A major impediment to mechanistic research in this regard has been the difficulty in infecting mouse models with SARS-CoV-2. Here, we utilized the mouse-adapted SARS-CoV-2 (strain MA10) that was plaque purified, grown in Vero-TMPRSS2 cells, and sequenced to ensure that it lacks the attenuating tissue culture adaptations present in stocks of the virus grown in standard Vero cells, the most commonly used cell line for SARS-CoV-2 propagation. Our extensive purification regimen allowed us to achieve measurable pathogenicity in C57BL/6 mice and to infect gene knockout animals for mechanistic research in vivo. Notably, mouse-adapted SARS-CoV-2 strains are emerging as models of choice for studies of viral pathogenicity in mice, though the lack of lethality in this model may not fully phenocopy fatal human disease. The K18-hACE2 mouse model also gained prominence early in the COVID-19 pandemic as an in vivo model susceptible to lethal SARS-CoV-2. However, in this system, hACE2 expression is driven by a keratin promoter, thus likely altering viral tropism, particularly in immune cell subsets where keratin promoter activity may not mirror endogenous ACE2 promoter activity. Thus, the mouse-adapted virus system employed in our studies is particularly advantageous for examining immune-mediated pathogenesis in the lung.

The active inflammasome complex has been implicated in many disease conditions and infections, including SARS-CoV-2. Cell culture experiments identified a minor role for the canonical inflammasome member caspase-1 (CASP1) in SARS-CoV-2 infection. CASP11, a member of the noncanonical inflammasome, has not been previously investigated in this context in vitro or in vivo. CASP11 is weakly expressed by resting cells, yet it is induced by bacterial infection and several cytokines. We mined available clinical data and found that the expression of human CASP4 and CASP5 in COVID-19 testing swab material correlates with the severity of SARS-CoV-2 infection. Additionally, we found that the expression of CASP4 is elevated in lung sections of SARS-CoV-2 patients. Similarly, mouse CASP11 is up-regulated in the lungs of WT mice in response to SARS-CoV-2. CASP11 restricts Legionella pneumophila and Burkholderia cenocepacia infections by regulating actin dynamics. CASP11 recognizes bacterial LPS in the cytosol, leading to downstream activation of CASP1 and IL-103. However, the role of CASP11 is not restricted to gram-negative bacteria that produce LPS, since we found that CASP11 is exploited by the gram-positive bacteria methicillin-resistant Staphylococcus aureus to survive in macrophages. In these cases, CASP11 regulates the functions of actin machinery to affect vesicular trafficking and cell migration. While it is possible that reduced neutrophil infiltration in SARS-CoV-2-infected Casp11−/− lungs is due to reduced cytokine and chemokine levels in the lungs, even with exogenous addition of chemoattractants, Casp11−/− immune cells, particularly neutrophils, fail to travel to the inflammation site due to an inherent defect in cell movement. Our lung histology and flow cytometry data show that neutrophil reduction in Casp 1−/− and Gsdmd−/− mice is comparable, yet the pathology in these animals is different. Our transcriptional profiling revealed a defect in cytokine responses, cellular recruitment, and immune activation in the absence of CASP11, demonstrating that Casp11−/− neutrophils can be nonfunctional when compared to WT and Gsdmd−/− neutrophils, a notion that is supported by the lack of NETosis in Casp11−/− neutrophils. On the other hand, GSDMD, which is considered the best characterized effector of CASP11 and CASP4, did not contribute to the lung pathology of SARS-CoV-2-infected mice, explaining why clinical trials using GSDMD inhibitors were not successful. Hence, our data indicate that CASP11 mediates many functions that are not executed by GSDMD.

While CASP4/11 can recognize cytosolic LPS from bacteria, it is also activated by oxidized phospholipids that are produced in damaged tissues. Overall oxidative stress, including oxidized phospholipids, are reportedly up-regulated in COVID-19 patients. Remarkably, oxidized phospholipids induce a CASP4/11-dependent cytokine response without inducing GSDMD-dependent cell death, thus mirroring our in vivo results in which Casp11−/− mice fare better during infection while Gsdmd−/− mice do not. High expression of CASP4/11 can result in its auto-activation in the absence of a ligand, as is observed upon its overexpression in vitro. Indeed, CASP4/11 levels can be highly induced by multiple cytokines, including IFNs, present in the SARS-CoV-2-infected lung.

The lungs of human patients infected with SARS-CoV-2 show diffuse immune cell infiltration, alveolar damage, alveolar edema and proteinaceous exudates, and destruction of endothelial cells, indicative of acute respiratory distress syndrome. Similar findings are detected in WT and Gsdmd−/− mice, while lung morphology appears healthier in Casp11−/− mice after SARS-CoV-2 infection. In addition, there is less weight loss, with fast recovery to normal weight, in Casp-11−/− mice compared with WT and Gsdmd−/− mice, which are slower to recover. Importantly, the differences in disease severity are not due to changes in viral burden among different genotypes. This is consistent with a lack of changes in global ISG expression in WT versus Casp11−/− or Gsdmd−/− lungs, which are genes implicated in viral resistance and clearance. Instead, we observed reduced inflammation and lung pathology dependent on CASP11 irrespective of viral loads. In Casp11−/− but not Gsdmd−/− SARS-CoV-2-infected mice, chemokines including Cxcl1, Cxcl2, and Cxcl14, which are involved in neutrophil and monocyte recruitment, were significantly down-regulated. However, there was no significant difference in expression of IL-1β between Casp11−/− and Gsdmd−/− mice. In vitro, IL-1β was barely detectable in the supernatants of macrophages infected with SARS-CoV-2. This can be explained by a recent publication demonstrating that SARS-CoV-2 nucleocapsid inhibits the cleavage of GSDMD in infected cells and hence prevents the release of IL-1β. In addition, our data demonstrate that IL-6 is elevated in infected lungs in a CASP11-dependent manner. IL-6 was identified during the COVID-19 pandemic as being a highly up-regulated mediator of disease severity in ill patients. Moreover, high levels of IL-6 can also activate the coagulation system and increase vascular permeability.

Postmortem studies have highlighted disseminated microthrombi that, together with increased mortality, morbidity, and long-term sequelae from SARS-CoV-2 infection, are considered hallmarks of severe COVID-19. Currently, the administration of an anticoagulant such as heparin for all hospitalized COVID-19 patients is associated with lower mortality rates and better prognosis. Typically, endothelial activation and damage lead to increased VWF production, and this activates the coagulation cascade, along with extensive NETosis elicited by neutrophils, leading to prothrombotic events. Importantly, we found here that lungs from Casp11−/− mice accumulate significantly less VWF in response to SARS-CoV-2 infection, which is largely confined to what appears to be the lining of blood vessels. In contrast, the distribution of VWF in infected WT lungs was intense and diffuse, indicating the presence of vascular damage.

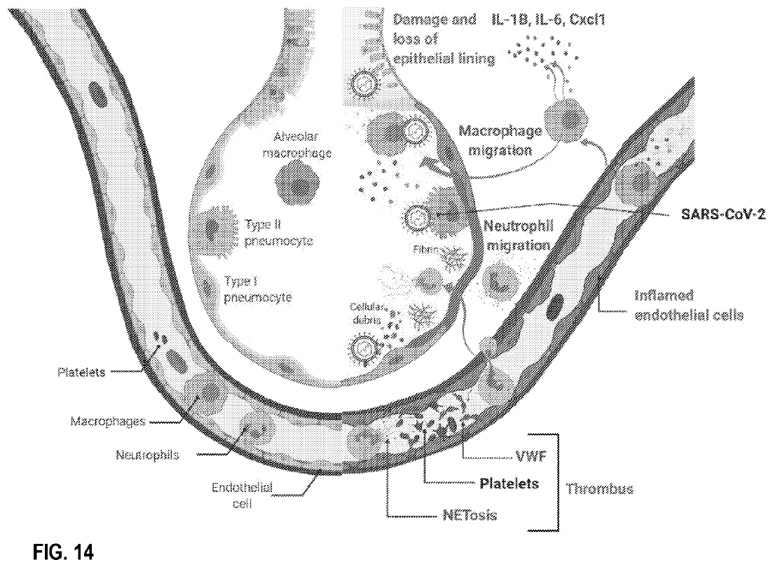

To further evaluate endothelial damage, we determined the expression of the transcription factor KLF2. Recent reports have linked the vascular injury that is associated with SARS-CoV-2 to reduction in the expression of KLF2 in lung endothelial cells. We found that KLF2 levels are largely preserved in Casp11−/− lungs but are significantly reduced in WT and Gsdmd−/− lungs. Moreover, the vascular abnormalities we detected on lung vascular tracing indicate severe endothelial damage and endothelialitis in WT SARS-CoV-2-infected lungs. These vascular features resemble the intussusceptive angiogenesis that has been described in SARS-CoV-2-infected human lungs. Importantly, the inhibition of angiogenesis through targeting VEGF has been proven beneficial in patients with severe SARS-CoV-2. Notably, we have found less expression of VEGF receptor 1 (FLT1) with less angiogenesis and neovascularization, which is often induced by hypoxia, in the infected Casp11−/− lungs compared to WT and Gsdmd−/− lungs. Our data demonstrate a previously unrecognized function for CASP11 in the promotion of coagulation pathways and endothelial dysfunction that lead to thrombotic events. Together with VWF, D-dimer and complement activation status are considered prognostic indicators of adverse outcomes of COVID-19. Remarkably, these factors emerge from the activation of multiple immunothrombosis-related pathways that we found are governed by CASP11 (FIG. 14). Therefore, targeting CASP11 can be beneficial in preventing cytokine storm and many of the thrombotic complications associated with SARS-CoV-2. Additionally, it is possible that exuberant CASP4/11 expression can serve as an earlier biomarker than those currently available for predicting severe disease, cytokine storm, and thrombosis. It is also important to note that unique pathogenic mechanisms can be at play among individuals experiencing severe COVID-19.

The findings collectively indicate that targeting the CASP11 homolog, human CASP4, during COVID-19 can prevent severe pneumonia, inflammation, tissue damage, and thrombosis as well as accompanying repercussions such as low oxygen, lung failure, need for ventilators, and possibly long-term sequelae. These advantageous effects can be achieved without compromising viral clearance. Targeting CASP4 alone can achieve benefits that exceed and replace the administration of a large number of individual anti-inflammatory agents and anti-thrombotics given to SARS-CoV-2 patients. Further research is needed to develop therapeutics in this regard.

### c) Materials and Methods

### (1) Biosafety.

All experiments with live SARS-CoV-2 were performed in The Ohio State University (OSU) BSL3 biocontainment facility. All procedures were approved by the OSU BSL3 Operations/Advisory Group, the OSU Institutional Biosafety Officer, and the OSU Institutional Biosafety Committee.

### (2) Viruses and Titers.

Mouse-adapted SARS-CoV-2 variant strain MA10, generated by the laboratory of Ralph Baric (University of North Carolina, Chapel Hill, NC) was provided by BEI Resources (Cat. #NR-55329). SARS-CoV-2 strain USA-WA1/2020 was also provided by BEI Resources (Cat. #NR-52281). Viral stocks from BEI Resources were plaque purified on Vero E6 cells to identify plaques lacking mutations in the polybasic cleavage site of the Spike protein via sequencing. Nonmutated clones were propagated on Vero E6 cells stably expressing TMPRSS2 (provided by Shan-Lu Liu, OSU, Columbus, OH) Virus aliquots were flash frozen in liquid nitrogen and stored at −80° C. Virus stocks were sequenced to confirm a lack of tissue culture adaptation in the polybasic cleavage site. Virus stocks and tissue homogenates were titered on Vero E6 cells.

### (3) Mice.

C57BL/6 WT mice were obtained from The Jackson Laboratory. Casp11^{-/-} mice were generously given by Dr. Junying Yuan at Harvard Medical School, Boston, MA. Gsdmd^{-/-} mice were a gift from Thirumala-Devi Kanneganti at St. Jude Children's Research Hospital, Memphis, TN. K18-hACE2 mice were purchased from The Jackson Laboratory. All infections were performed intranasally on anesthetized mice with viruses diluted in sterile saline. All mice were housed in a pathogen-free facility, and experiments were conducted with approval from the Animal Care and Use Committee at OSU (Columbus, OH), which is accredited by the American Association for Accreditation of Laboratory Animal Care International according to guidelines of the Public Health Service as issued in the Guide for the Care and Use of Laboratory Animals.

### (4) Derivation of Single-Cell Suspension and Primary Lung Macrophages.

Lungs were perfused with cold phosphate-buffered saline (PBS) to remove circulating intravascular white blood cells. Lungs were dissected into single lobes before being dissociated into single-cell suspension using gentleMACS Octo Dissociator and Miltenyi lung dissociation kit (Miltenyi Biotec, 130-095-927). Red blood cells (RBCs) were lysed by incubating cells in 2 mL ACK buffer for 5 min at room temperature (RT). After RBC lysis, cells were washed in DPBS containing 1% bovine serum albumin (BSA). The single-cell suspension was centrifuged, and the cell pellets were washed twice with PBS. Cell pellets were further suspended in 0.5 mL PBS 1% BSA. This was followed by CD11b magnetic bead (Miltenyi Biotec, 130-049-601) isolation technique to positively select for macrophages expressing the pan-macrophage/monocyte CD11b marker.

### (5) Flow Cytometry.

Single-cell suspension from the previous step was stained with fluorophore-conjugated antibodies for fluorometric analysis as described before.

### (6) Murine Tracheobronchial Epithelial Three-Dimensional (3D) Cultures.

Murine trachea and bronchioles were dissected from two mice each of C57BL/6 WT, Casp11^{-/-}, and Gsmd^{-/-}. Isolation of tracheobronchial epithelial cells was as follows.

Tissues were washed, and tracheas were incubated overnight in Ham's F-12, 1% penicillin/streptomycin, 1% amphotericin B (Thermo Fisher Scientific, #15290018) and Pronase from Streptomyces griseus (Sigma-Aldrich, #10165921001) solution. Digestion of trachea and bronchioles were neutralized with 10% fetal bovine solution (FBS; Life Technologies, #10438026), and tracheal airway cells were gently scraped. Cells were washed three times in Ham's F-12, 10% FBS, and 1% penicillin/streptomycin solution and further digested in deoxyribonuclease I solution (Sigma-Aldrich, #DN25-10) in Ham's F-12 with 10 mg/mL BSA (Thermo Fisher Scientific, #BP9706). Airway cells were then washed with Murine Tracheobronchial Epithelial Cell (MTEC) base medium [1:1 Ham's F-12: Dulbecco's modified Eagle's medium (Thermo Fisher Scientific, #11995065), plus 10% FBS, 1% penicillin/streptomycin, 50 μg/mL gentamicin (Life Technologies, #15710064), and 0.03% wt/vol NaHCO₃]. Cells were plated in a T25 flask (Thermo Fisher Scientific, #1012610) overnight in MTEC medium at 37° C., 5% CO₂. The next day, medium was switched to 1:1 of MTEC and PneumaCult-Ex PLUS medium (StemCell Technologies, #05040) and fed every other day until expansion of cells to ˜80% confluent. Epithelial cells were then trypsinized twice with TrypLE Express (Thermo Fisher Scientific, #12605010) to remove residual fibroblast cells and seeded at a density of 50,000 cells per transwell in Corning 6.5-mm, 24-well transwells (Thermo Fisher Scientific, #07200154) in 1.1 MTEC:PneumaCult-Ex PLUS medium. Cells were fed for 4 to 5 d until airlifted and continued to be grown at air-liquid interface (ALI) with PneumaCult ALI medium (StemCell Technologies, #05001) until fully differentiated (4 wk).

### (7) Immunoblotting.

Protein extraction from lung tissue was performed using TRIzol reagent (Thermo Fisher Scientific, #15596026) according to the manufacturer's instructions. Equal amounts of protein were separated by sodium dodecyl sulfate-polyacrylamide gel electrophoresis and transferred to a polyvinylidene fluoride membrane. Membranes were incubated overnight with antibodies against CASP11 (Cell Signaling Technology, 14340), VWF (Protein Tech, I1778-1-AP), and β-Actin (Cell Signaling Technology, 3700). Corresponding secondary antibodies conjugated with horseradish peroxidase in combination with enhanced chemiluminescence reagent (Amersham, RPN2209) were used to visualize protein bands Densitometry analyses were performed by normalizing target protein bands to their respective loading control (β-Actin) using ImageJ software.

### (8) ELISAs.

Cytokine/chemokine ELISAs were performed on lung homogenates or macrophage supernatants using R&D Systems Duoset ELISA kits (IL-6, DY406; IL-1b, DY401; CXCL1, DY453) according to the manufacturer's instructions.

### (9) Histology.

Lungs were removed from infected mice and fixed in 10% formalin at RT. Sample preparation, processing, H&E staining, and semiquantitative slide evaluation using ordinal grading scales were performed. Lungs used for immunofluorescence (IF) staining and RNAscope ISH technique were embedded in optimal cutting temperature compound (OCT) and flash frozen, while lung tissue used for immunohistochemistry (IHC) was embedded in paraffin blocks.

### (10) IHC and IF Staining for Mouse Tissues.

IF staining of mouse lung sections was performed. Slides were washed three times for 15 min with PBS to remove residual OCT The sections were then incubated in the blocking solution (PBS containing 10% donkey serum; MilliporeSigma, Cat. #S30-100 mL), 2% BSA (Thermo Fisher Scientific, BP1600-100), and 0.3% Triton X-100 (Thermo Fisher Scientific, BP151-100) for 2 h at RT. Sections were then transferred to blocking solution containing the primary antibody against IL-1β (GeneTex, GTX74034) and incubated overnight at 4° C. After that, sections were washed with PBS 3× for 15 min each. Then, they were incubated with the blocking solution containing the secondary antibody for 2 h at RT DAPI (Thermo Fisher Scientific, Cat. #D1306) was added to the staining solution in the last 15 min of incubation at a final concentration (5 μg/mL). Finally, sections were washed with PBS 3× for 15 min. Antifade mounting media (Thermo Fisher Scientific, Cat #P36934) was added before coverslipping. For IHC, Ly6G (Abcam, ab25377) and SARS-CoV-2 nucleocapsid protein (GeneTex, GTX635686) primary antibodies were used. All the stainings were performed at Histowiz, Inc., Brooklyn, NY, using the Leica Bond RX automated stainer (Leica Microsystems) The slides were dewaxed using xylene and alcohol-based dewaxing solutions. Epitope retrieval was performed by heat-induced epitope retrieval of the formalin-fixed, paraffin-embedded tissue using citrate-based pH 6 solution for 40 min at 95° C. The tissues were first incubated with peroxide block buffer (Leica Microsystems), followed by incubation with the rabbit Caspase 4 antibody (Novus Bio, NBP1-87681) at 1:700 dilution for 30 min, followed by diaminobenzidine (DAB) rabbit secondary reagents: polymer, DAB refine, and hematoxylin (Leica Microsystems). The slides were dried, coverslipped, and visualized using a Leica Aperio AT2 slide scanner (Leica Microsystems).

### (11) RNAscope ISH.

Lung tissue was fixed and embedded in OCT as described above. Sections of 15-μm thickness were mounted on Plus charged slides. ISH was performed using RNAscope Multiplex Fluorescent Reagent Kit v2 (Advanced Cell Diagnostics, Cat. #323100) as described before. All incubations between 40 and 60° C. were conducted using an ACD HybEZ II Hybridization System with an EZ-Batch Slide System (Advanced Cell Diagnostics, Cat. #321710). Slides were washed in PBS twice to remove any residual OCT, then baked at 60° C. for 30 min. Baked slides were subsequently postfixed in cold 10% formalin for 15 min, then washed and treated with hydrogen peroxide solution (10 min at RT, Advanced Cell Diagnostics, Cat. #322335). After being rinsed twice with double-distilled H2O, sections were incubated in RNAscope Target Retrieval Solution (98° C. for 5 min, Advanced Cell Diagnostics, Cat. #322001) and rinsed three times. Next, a hydrophobic barrier was created around the tissue using an ImmEdge Pen (Advanced Cell Diagnostics; Cat #310018), and slides were incubated with RNAscope Protease III (30 min at 40° C., Advanced Cell Diagnostics, Cat #322337) and subsequently incubated with RNAscope target probes VWF (Cat. #499111), FLT1 (Cat. #415541-C2), and Casp4/Casp11 (Cat #589511) for 2 h at 40° C. Next, slides were washed twice with 1× Wash Buffer (Advanced Cell Diagnostics, Cat. #310091; 2 min/rinse at RT), followed by sequenmial tissue application of the following RNAscope Multiplex FL v2 Amp 1 (Advanced Cell Diagnostics, Cat. #323101). RNAscope Multiplex FL v2 Amp 2 (Advanced Cell Diagnostics, Cat. #323102), and RNAscope Multiplex FL v2 Amp 3 (Advanced Cell Diagnostics, Cat. #323103). This was followed by application of RNAscope Multiplex FL v2 HRP C1 or C2 (IS min at 40° C.; Advanced Cell Diagnostics, Cat. #323104). Finally, Opal dyes (Thermo Fisher Scientific, NC1601877 and NC601878) were then applied, after being (Thermo Fisher Scientific, NC1601877) diluted in RNAscope TSA buffer, (Advanced Cell Diagnostics, Cat. #322809) for 30 min at 40° C. HRP blocker was subsequently added to halt the reaction. Finally, slides were incubated with DAPI, coverslipped with ProLong Gold Antifade Mountant (Thermo Fisher Scientific, P36930), and stored at 4° C. until image acquisition.

### (12) Confocal Imaging and Analysis.

Fluorescent images were captured on an Olympus FV 3000 inverted microscope with a motorized stage. A 2× objective was used to create a map of the lung section in the X,Y dimension. This was followed by using 20× objective to create a stitched z-stacked 3D panoramic view of the lung section. Images were taken by using the 488-nm, 543-nm, and 405-nm (for DAPI) lasers. Image reconstructions of z-stacks and intensity projection images were generated in IMARIS software (Bitplane, Inc.). Ftl1 mRNA expression was quantified using spot function in IMARIS Number of cells was also quantified via the spot functions.

(13) Vasculature Labeling with Conjugated Albumin.

The mouse vasculature was labeled Briefly, mice were transcardially perfused with 10% formalin in PBS. Mice were then perfused with 5 mL 0.05% albumin-tetramethylrhodamine isothiocyanate bovine (Sigma-Aldrich, #A2289) in 2% gelatin from porcine skin (Sigma-Aldrich, #(G1890). At the time of injection, the temperature of the gel solution was kept at 45° C. After the heart was clamped, mice were placed on ice to lower the body temperature and allow gel formation. Lungs were postfixed in 10% formalin for 10 d. The unsectioned lungs were then cleared using the advanced CUBIC (Clear, Unobstructed Brain Imaging Cocktails and Computational Analysis) protocol and imaged using a confocal microscope (C2, Nikon).

### (14) NET Formation Assay.

Bone marrow was collected from WT, Gsdmd^{-/-}, or Casp1^{-/-} mice, then neutrophils were negatively selected by using the EasySep mouse neutrophil enrichment kit (STEM Cell Technologies, #19762A) and 200,000 neutrophils/well were plated in a 24-well plate on fibronectin-coated glass coverslip. Polymorphonuclear neutrophils were stimulated for 4 h with 100 nM PMA (Sigma-Aldrich, #P8139-10MG) or conditioned media from SARS-CoV-2-infected epithelial cells. The cells were fixed with 4% paraformaldehyde, permeabilized with 0.2% Triton X-100 for 10 min, and blocked with 10% goat serum for 30 min at RT. For the visualization of NETs, neutrophils were stained with rabbit anti-mouse Histone 2b (Abcam, #ab1790), mouse anti-double-stranded DNA (dsDNA) (Abcam, #ab27156), goat anti-rabbit immunoglobulin (lg)G Alexa Fluor 555 (Thermo Fisher Scientific, #A32732), goat anti-mouse IgG Alexa Fluor 488 (Abcam, #ab150113), and wheat germ agglutinin Alexa Fluor 350 (Thermo Fisher Scientific, #W11263). The coverslips were mounted with Fluoroshield Mounting Medium (Abcam, #ab104135). The cells were visualized by confocal microscopy (Zeiss 800 Confocal microscope) The % of cells producing NETs was calculated per field of view by using Fiji software (NIH). The cells producing NETs were counted when DNA was decondensed and ejected from the cells: % NETs=(neutrophds with DNA projections×100) total number of neutrophils.

### (15) RNA Sequencing and Data Analysis.

Total RNA was extracted from day 2 SARS-CoV-2 WT, Casp11^{-/-}, and Gsdmd^{-/-} infected lungs by TRIzol reagent (Thermo Fisher Scientific, #15596026) according to the manufacturer's instructions. RNA cleaning and concentration were done using Zymo Research, RNA Clean & Concentrator-5 kit (Cat. #R1015) following the manufacturer's protocol. Fluorometric quantification of RNA and RNA integrity analysis were carried out using RNA Biochip and Qubit RNA Fluorescence Dye (Invitrogen). Complementary DNA (cDNA) libraries were generated using NEBNext Ultra II Directional (stranded) RNA Library Prep Kit for Illumina (NEB, #E7760L). Ribosomal RNA was removed using NEBNext rRNA Depletion Kit (human, mouse, rat) (E #E6310X). Libraries were indexed using NEBNext Multiplex Oligos for Illumina Unique Dual Index Primer Pairs (NEB, #6440S/L) Library prep-generated cDNA was quantified and analyzed using Agilent DNA chip and Qubit DNA dye. Ribo-depleted total transcriptome libraries were sequenced on an Illumina NovaSeq SP flow cell (paired-end, 150-bp format; 35 to 40 million clusters, equivalent to 70 to 80 million reads). Library preparation, quality control, and sequencing were carried out at Nationwide Children's Hospital genomic core.

Sequencing data processing and analysis were performed by the Bioinformatics Shared Resource Group at OSU using previously published pipelines. Briefly, raw RNA sequencing data (fastq) were aligned to mouse reference genome (GRCh38) using hisat2 (v2.1.0) and converted to counts using the “subread” package (v1.5.1) in R. In the case of multimapped reads, the primary alignment was counted. Low expressed counts were excluded if more than half of the samples did not meet the inclusion criteria (2 counts per million [CPM]). Data were normalized using “voom,” and statistical analysis for differential expression was performed with “limma”. For data visualization, DESeq2 rlog transformation was used for principal component analysis (PCA). Volcano plots were generated with “EnhancedVolcano” and heatmaps were generated with ComplexHeatmap” using R. Functional enrichment was performed with Ingenuity Pathway Analysis (Qiagen) to enrich for IPA Canonical Pathways, “clusterProfiler” to generate enrichment maps, and EnrichR.

### (16) Single Cell RNA Sequencing Data Mining.

Human lung single-cell expression analysis for CASP4 and CASP5 was performed by the Human Protein Atlas version 21.0. Mouse lung single-cell expression analysis for Casp11 was performed on published data (GEO dataset accession No. GSE186360).

### (17) Statistical Analysis.

Data were analyzed using GraphPad Prism 8.3.0. All figures display mean and SD (SD) or SEM (SEM) from independent experiments, as indicated in the figure legends. Comparisons between groups were conducted with either upaired t test or ANOVA followed by Tukey's multiple comparisons test. Adjusted P<0.05 was considered statistically significant.

### D. REFERENCES

Ackermann, M., et al. Pulmonary Vascular Endothelialitis, Thrombosis, and Angiogenesis in Covid-19. N Engl J Med 383, 120-128 (2020).
Adil, M. T., et al. SARS-CoV-2 and the pandemic of COVID-19. Postgrad Med J 97, 110-116 (2021).
Akhter, A., et al. Caspase-11 promotes the fusion of phagosomes harboring pathogenic bacteria with lysosomes by modulating actin polymerization. Immunity 37, 35-47 (2012).
Badraoui, R., Alrashedi, M. M., El-May, M. V. & Bardakci, F. Acute respiratory distress syndrome: a life threatening associated complication of SARS-CoV-2 infection inducing COVID-19. J Biomol Struct Dyn, 1-10 (2020).
Bao, L., et al. The pathogenicity of SARS-CoV-2 in hACE2 transgenic mice. Nature 583, 830-833 (2020).
Broz, P., Pelegrin, P. & Shao, F. The gasdermins, a protein family executing cell death and inflammation. Nat Rev Immunol (2019).
Coperchini, F., Chiovato, L., Croce, L., Magri, F. & Rotondi, M. The cytokine storm in COVID-19: An overview of the involvement of the chemokine/chemokine-receptor system. Cytokine Growth Factor Rev 53, 25-32 (2020).
Cornelissen, A., et al. Effects of simulated COVID-19 cytokine storm on stent thrombogenicity. Cardiovasc Revasc Med (2021).
Das, A., Roy, S., Swarnakar, S. & Chatterjee, N. Understanding the immunological aspects of SARS-CoV-2 causing COVID-19 pandemic: A therapeutic approach. Clin Immunol, 108804 (2021).
Di Giovanna, A. P., et at. Whole-Brain Vasculature Reconstruction at the Single Capillary Level. Sci Rep 8, 12573 (2018).
Estfanous, S., et al. Elevated Expression of MiR-17 in Microglia of Alzheimer's Disease Patients Abrogates Autophagy-Mediated Amyloid-beta Degradation. Front Immunol 12, 705581 (2021).
Francischetti, I. M. B., et al. Upregulation of pulmonary tissue factor, loss of thrombomodulin and immunothrombosis in SARS-CoV-2 infection. EClinicalMedicine 39, 101069 (2021).
Gadepalli, V. S., Ozer, H. G., Yilmaz, A. S., Pietrzak, M. & Webb, A. BISR-RNAseq: an efficient and scalable RNAseq analysis workflow with interactive report generation. BMC Bioinformatics 20, 670 (2019).
Giardini, V., et al. Increased sFLT-1/PlGF ratio in COVID-19. A novel link to angiotensin II-mediated endothelial dysfunction. Am J Hematol 95, E188-E191 (2020).
Helms, J., et al. High risk of thrombosis in patients with severe SARS-CoV-2 infection: a multicenter prospective cohort study. Intensive Care Med 46, 1089-1098 (2020).
Junqueira, C., et al. SARS-CoV-2 infects blood monocytes to activate NLRP3 and AIM2 inflammasomes, pyroptosis and cytokine release. medRxiv (2021).
Kayagaki, N., et al. Caspase-11 cleaves gasdermin D for non-canonical inflammasome signalling. Nature 526, 666-671 (2015).
Kayagaki, N., et al. Non-canonical inflammasome activation targets caspase-11. Nature 479, 117-121 (2011).
Kim, D., Langmead, B. & Salzberg, S. L. HISAT: a fast spliced aligner with low memory requirements. Nat Methods 12, 357-360 (2015).
Krause, K., et al. CASP4/caspase-11 promotes autophagosome formation in response to bacterial infection. Autophagy 14, 1928-1942 (2018).
Krause, K., et al. Caspase-11 counteracts mitochondrial ROS-mediated clearance of Staphylococcus aureus in macrophages. EMBO Rep 20, e48109 (2019).
Kuleshov, M. V., et al. Enrichr: a comprehensive gene set enrichment analysis web server 2016 update. Nucleic Acids Res 44, W90-97 (2016).
Kyle Caution, et al. Caspase-11 mediates migration of neutrophils and necroptosis during acute gouty arthritis. Frontiers in Immunology Accepted(2019).
Ladikou, E. E., et al. Von Willebrand factor (vWF): marker of endothelial damage and thrombotic risk in COVID-19? Clin Med (Lond) 20, e178-e182 (2020).
Lavillegrand, J. R., et al. Elevated plasma IL-6 and CRP levels are associated with adverse clinical outcomes and death in critically ill SARS-CoV-2 patients: inflammatory response of SARS-CoV-2 patients. Ann Intensive Care 11, 9 (2021).
Lee, T. H., et al. SARS-CoV-2 infection reduces Kruppel-Like Factor 2 in human lung autopsy. bioRxiv (2021).
Leist, S. R., et al. A Mouse-Adapted SARS-CoV-2 Induces Acute Lung Injury and Mortality in Standard Laboratory Mice. Cell 183, 1070-1085 e1012 (2020).
Liao, Y., Smyth, G. K. & Shi, W. featureCounts: an efficient general purpose program for assigning sequence reads to genomic features. Bioinformatics 30, 923-930 (2014).
Liu, F., et al. SARS-CoV-2 Infects Endothelial Cells In Vivo and In Vitro. Front Cell Infect Microbiol 11, 701278 (2021).
Ma, J., et al. SARS-CoV-2 nucleocapsid suppresses host pyroptosis by blocking Gasdermin D cleavage. EMBO J, e108249 (2021).
Major, J., et al. Type I and III interferons disrupt lung epithelial repair during recovery from viral infection. Science 369, 712-717 (2020).
McCray, P. B., Jr., et al. Lethal infection of K18-hACE2 mice infected with severe acute respiratory syndrome coronavirus. J Virol 81, 813-821 (2007).
Melo Silva Junior, M. L., Souza, L. M. A., Dutra, R., Valente, R. G. M. & Melo, T. S. Review on therapeutic targets for COVID-19: insights from cytokine storm. Postgrad Med J 97, 391-398 (2021).
Merkley, C. M., Shuping, S. L., Sommer, J. R. & Nestor, C. C. Evidence That Agouti-Related Peptide May Directly Regulate Kisspeptin Neurons in Male Sheep. Metabolites 11(2021).
Middleton, E. A., el al. Neutrophil extracellular traps contribute to immunothrombosis in COVID-19 acute respiratory distress syndrome. Blood 136, 1169-1179 (2020).
Moreau, G. B., et al. Evaluation of K18-hACE2 Mice as a Model of SARS-CoV-2 Infection. Am J Trop Med Hyg 103, 1215-1219 (2020).
Oh, C., Verma, A. & Aachoui, Y. Caspase-11 Non-canonical Inflammasomes in the Lung. Front Immunol 11, 1895 (2020).
Pang, J., et al. Efficacy and tolerability of bevacizumab in patients with severe Covid-19. Nat Commun 12, 814 (2021).
Radermecker, C., et al. Neutrophil extracellular traps infiltrate the lung airway, interstitial, and vascular compartments in severe COVID-19. J Exp Med 217(2020).
Ritchie, M. E., et al. limma powers differential expression analyses for RNA-sequencing and microarray studies. Nucleic Acids Res 43, e47 (2015).
Rodrigues, T. S., et al. Inflammasomes are activated in response to SARS-CoV-2 infection and are associated with COVID-19 severity in patients. J Exp Med 218(2021).
Rodriguez, C., et al. Pulmonary Endothelial Dysfunction and Thrombotic Complications in Patients with COVID-19. Am J Respir Cell Mol Biol 64, 407-415 (2021).
Ruifrok, A. C. & Johnston, D. A. Quantification of histochemical staining by color deconvolution. Anal Quant Cytol Histol 23, 291-299 (2001).
Sasaki, M., et al. SARS-CoV-2 variants with mutations at the S1/S2 cleavage site are generated in vitro during propagation in TMPRSS2-deficient cells. PLoS Pathog 17, e1009233 (2021).
Susaki, E. A., et al. Advanced CUBIC protocols for whole-brain and whole-body clearing and imaging. Nat Protoc 10, 1709-1727 (2015).
Vanaja, S. K., et al. Bacterial Outer Membrane Vesicles Mediate Cytosolic Localization of LPS and Caspase-11 Activation. Cell 165, 1106-1119 (2016).
Varga, Z., et al. Endothelial cell infection and endotheliitis in COVID-19. Lancet 395, 1417-1418 (2020).
Yi, Y. S. Caspase-11 non-canonical inflammasome: a critical sensor of intracellular lipopolysaccharide in macrophage-mediated inflammatory responses. Immunology 152, 207-217 (2017).
Zakeri, A., Jadhav, A. P., Sullenger, B. A. & Nimjee, S. M. Ischemic stroke in COVID-19-positive patients: an overview of SARS-CoV-2 and thrombotic mechanisms for the neurointerventionalist. J Neurointerv Surg 13, 202-206 (2021).

1. A method of treating a coronaviral infection or the symptoms or disease state thereof comprising administering to a subject with a coronaviral infection an inhibitor of caspase 4 (CASP4).

2. The method of claim 1, wherein the inhibitor of CASP4 comprises an antibody, siRNA, shRNA, protein, or small molecule.

3. The method of claim 1, wherein the coronaviral infection is a SARS-CoV2 infection.

4. The method of claim 1, wherein the disease state is COVID-19.

5. The method of claim 1, wherein the symptoms comprise thrombosis, pneumonia, inflammation, and tissue damage.

6. A method of assessing the severity of a SARS-CoV-2 infection comprising:
a) obtaining a pulmonary tissue sample from the subject; and
b) measuring the level of CASP4 expression in the sample;
wherein the level of CASP4 directly correlates with severity of infection.

7. The method of claim 6, wherein CASP4 expression level is measured by western blot, quantitative western blot, RNA sequencing (RNAseq), Single Cell RNA Sequencing, (scRNAseq), high-performance liquid chromatography (HPLC), liquid chromatography-mass spectrometry (LC/MS), enzyme linked immunosorbent assay (ELISA), immunoelectrophoresis, or protein array.

8. The method of claim 6 further comprising administering to the subject an inhibitor of CASP4, von Willebrand factor (vWF), IL-1β, or Cxcl1.
